(19) United States
(12) Patent Application Publication
(10) Pub. No.: US 2026/0347759 A1
(43) Pub. Date: Nov. 5, 2026

(54) BIFIDOBACTERIUM STRAINS WITH IMPROVED STABILITY

(71) Applicant: CHR. HANSEN A/S, Hoersholm (DK)

(72) Inventors: Zeidan AHMAD, Hoersholm (DK); Marie SCHOEPPING, Hoersholm (DK)

(73) Assignee: CHR. HANSEN A/S, Hoersholm (DK)

(21) Appl. No.: 19/106,313

(22) PCT Filed: Aug. 25, 2023
(86) PCT No.: PCT/EP2023/073415

§ 371 Date: Feb. 25, 2025

(30) Foreign Application Priority Data
Aug. 25, 2022 (EP) 22192161.2

(51) Int. Cl. C12N 1/205; A61K 35/745; A61K 47/26; C12N 9/10; C12R 1/01

(52) U.S. Cl. CPC C12N 1/205; A61K 35/745; A61K 47/26; C12N 9/1029; C12Y 203/01085; C12R 2001/01

(57) ABSTRACT

The present invention relates to mutated strains of Bifidobacterium longum with improved properties, such as stability. Further, the invention relates to a method for manufacturing mutated strains of Bifidobacterium longum, as well as use of these strains for improving the intestinal barrier function and/or eliciting an anti-inflammatory immune response.

### TECHNICAL FIELD

The present invention relates to mutated strains of Bifidobacterium longum with improved properties, such as stability.

Further, the invention relates to a method for manufacturing mutated strains of Bifidobacterium longum, as well as use of these strains for improving the intestinal barrier function and/or eliciting an anti-inflammatory immune response.

### BACKGROUND

Bifidobacteria are natural inhabitants of the gastrointestinal tract possessing genetic adaptations that enable colonization of this harsh and complex habitat. Bifidobacteria interact with key elements of intestinal functioning and contribute to maintaining homeostasis. Recent scientific progress has demonstrated that bifidobacteria, through strain-dependent interactions with the host may reduce mucosal antigen load, improve the intestinal barrier, and induce regulation of local and systemic immune responses.

Due to their recognized benefits to human health, bifidobacteria are used as probiotics. Probiotics are “live micro-organisms which, when administered in adequate amounts, confer a health benefit on the host” (FAO/WHO, 2001). About a dozen Bifidobacterium strains with clinically documented effects are commercially available. Half of these are Bifidobacterium animalis subsp. lactis strains and the remaining are Bifidobacterium longum subsp. longum, B. longum subsp. infantis, B adolescentis, or Bifidobacterium breve strains.

EP 0 768 375 describes specific strains of Bifidobacterium subspecies, that are capable to become implanted in the intestinal flora and being capable to competitively exclude adhesion of pathogenic bacteria to intestinal cells. These Bifidobacteria are reported to assist in immunomodulation and thus in the maintenance of the individual's health. The immunomodulation effect of Bifidobacteria may even be conferred onto unborn children. WO 01/97822 e.g. describes that intake of Bifidobacterium animalis strain BB-12® by the mother during her pregnancy reduces the occurrence of atopic diseases in children. Also WO 03/099037 describes that Bifidobacterium animalis strain BB-12® are able to beneficially modify the immune response. According to Masco et al. (2004) Bifidobacterium animalis strain BB-12® should correctly be referred to as Bifidobacterium animalis subsp. lactis strain BB-12®.

The Bifidobacterium animalis subsp. lactis strain BB-12® is relatively stable. This enables the strain to be conveniently incorporated into a number of products.

However, there remains a need for other Bifidobacterium strains with high stability profile.

### SUMMARY OF THE INVENTION

The invention is related to a variant of Bifidobacterium with a single nucleotide polymorphism in its multifunctional microbial type I fatty acid synthase (FAS) gene.

This mutation is associated with the ability of the variant to grow in the absence of a fatty acid source, such as Polysorbate/Tween® 80, and contributes to improved robustness and stability of the variant as compared to its parental strain.

The variant may be a Bifidobacterium longum strain with a mutation in the gene resulting in an amino acid substitution at position 407 of the protein sequence. The wild type arginine in this position, is replaced with another small, uncharged amino acid, such as glycine or serine.

According to a first aspect, an isolated strain of Bifidobacterium longum is provided, characterized in that the arginine in the FAS gene is substituted by an amino acid that is glycine or serine.

In one embodiment, the isolated strain of Bifidobacterium longum is characterized in that the FAS gene sequence comprises SEQ ID NO: 1; and it has ability to grow in the absence of an exogenous fatty acid source.

In one embodiment, the isolated strain of Bifidobacterium longum is characterized in that the FAS protein sequence comprises SEQ ID NO: 2; and it has ability to grow in the absence of an exogenous fatty acid source.

In one embodiment, the isolated strain has a low ratio of unsaturated to saturated fatty acids as compared to the wild type strain, such as around 1:1.

In one embodiment, the strain is selected from the group consisting of B. longum subsp. longum BB-46, B. longum subsp. longum AF13-34 or B. longum subsp. longum AF13-41.

In one embodiment, the strain is the B. longum subsp. longum BB-46.

According to a second aspect, a probiotic product is provided, comprising an isolated strain according to any one of claims 1 to 6 and a cryoprotectant.

In one embodiment, the cryoprotectant is a saccharide or a sugar alcohol.

According to a third aspect, an isolated strain according to the first aspect or a probiotic product according to the second aspect is provided for use in prevention, alleviation of symptoms, and/or treatment of an intestinal inflammatory condition such as IBD and IBS, a liver disease such as NAFLD, NASH, cirrhosis, or alcohol-related liver disease, a metabolic disorder such as metabolic syndrome, insulin resistance, type 2 diabetes, obesity, cardiovascular atherosclerosis, an autoimmune disease, such as celiac disease, type 1 diabetes, multiple sclerosis or rheumatoid arthritis, and/or a mental condition such as major depressive disorder, a mood disorder, a cognitive disorder, chronic fatigue syndrome, or anxiety.

According to a fourth aspect, a method of manufacturing a Bifidobacterium longum strain is provided, wherein the growth medium is a medium without an exogenous fatty acid source, such as Tween® 80.

According to a fifth aspect, a method is provided for the prevention, alleviation of symptoms, and/or treatment of an intestinal inflammatory condition such as IBD and IBS, a liver disease such as NAFLD, NASH, cirrhosis, or alcohol-related liver disease, a metabolic disorder such as metabolic syndrome, insulin resistance, type 2 diabetes, obesity, cardiovascular atherosclerosis, an autoimmune disease, such as celiac disease, type 1 diabetes, multiple sclerosis or rheumatoid arthritis, and/or a mental condition such as major depressive disorder, a mood disorder, a cognitive disorder, chronic fatigue syndrome, or anxiety, the method comprising administering a therapeutically effective dose of an isolated strain according the first aspect or a probiotic product according to the second aspect to an individual in need thereof.

### DETAILED DESCRIPTION

The present invention relates to an isolated strain of B. longum, such as B. longum subsp. longum characterized in that a) the strain comprises a nucleic acid sequence according to SEQ ID NO: 1; or b) sequences which encode proteins having at least 90% identity to SEQ ID NO: 2, and an amino acid mutation at position 407 of SEQ ID NO: 2.

In specific embodiments, the strain comprises nucleic acid sequences which encode proteins having at least 90% identity to SEQ ID NO 2, such as sequences encoding proteins having at least 91% identity, at least 92% identity, at least 93% identity, at least 94% identity, at least 95% identity, at least 96% identity, at least 97% identity, at least 98% identity, or at least 99% identity to SEQ ID NO 2. In one embodiment, the strain includes a nucleotide sequence which encodes an amino acid sequence of SEQ ID NO 2.

In one specific embodiment, the amino acid at position 407 of SEQ ID NO: 2 is glycine or serine.

For purposes of the present invention, the degree of sequence identity between two nucleotide sequences or two amino acid sequences is determined using the sequence alignment method of Clustal Omega for nucleotide sequence (DNA) or amino acid sequence (protein), respectively, pairwise alignment.

A bacterial “strain” as used herein refers to a bacterium which remains genetically unchanged when grown or multiplied. The multiplicity of identical bacteria is included. “Wild type strain” refers to the non-mutated form of a bacterium, as found in nature.

In the present context, the term “derived strain” should be understood as a strain derived from a wild type strain by means of e.g. genetic engineering, radiation and/or chemical treatment, and/or selection, adaptation, screening, etc. In specific embodiments the derived strain is a functionally equivalent mutant, e.g. a mutant that has substantially the same, or improved, properties (e.g. regarding probiotic properties) as the mother strain. Such a derived strain is a part of the present invention. The term “derived strain” includes a strain obtained by subjecting a strain of the invention to any conventionally used mutagenesis treatment including treatment with a chemical mutagen such as ethane methane sulphonate (EMS) or N-methyl-N′-nitro-N-nitroguanidine (NTG), UV light or to a spontaneously occurring mutant.

A “mutant bacterium” or a “mutant strain” refers to a natural (spontaneous, naturally occurring) mutant bacterium or an induced mutant bacterium comprising one or more mutations in its genome (DNA) which are absent in the wild type DNA. It can also be a single-nucleotide polymorphisms (SNP). An “induced mutant” is a bacterium where the mutation was induced by human treatment, such as treatment with any conventionally used mutagenesis treatment including treatment with chemical mutagens, such as a chemical mutagen selected from (i) a mutagen that associates with or become incorporated into DNA such as a base analogue, e.g. 2-aminopurine or an interchelating agent such as ICR-191, (ii) a mutagen that reacts with the DNA including alkylating agents such as nitrosoguanidine or hydroxylamine, or ethane methyl sulphonate (EMS) or N-methyl-N′-nitro-N-nitroguanidine (NTG), UV- or gamma radiation etc. In contrast, a “spontaneous mutant” or “naturally occurring mutant” has not been mutagenized by man.

A variant strain, such as a mutant, may have been subjected to several mutagenesis treatments (a single treatment should be understood as one mutagenesis step followed by a screening/selection step), but typically no more than 20, no more than 10, or no more than 5 treatments are carried out. In specific embodiments of derived strains, such as mutants, less than 1%, less than 0.1%, less than 0.01%, less than 0.001% or even less than 0.0001% of the nucleotides in the bacterial genome have been changed (such as by replacement, insertion, deletion or a combination thereof) compared to the mother strain.

Mutant bacteria as described above are non-GMO, i.e. not genetically modified by recombinant DNA technology. As an alternative to above preferred method of providing the mutant by random mutagenesis, it is also possible to provide such a mutant by site-directed mutagenesis, e.g. by using appropriately designed PCR techniques or by using a transposable element which is integratable in bacterial replicons, or by genome editing.

When the mutant is provided as a spontaneously occurring mutant the above wild type strain is subjected to the selection step without any preceding mutagenesis treatment.

A mutant strain of any of the B. longum strains with accession number DSM 15955 can be obtained by subjecting the strain to mutagenesis treatment as described to obtain mutant strains and selecting for mutant strains having the desired properties. Alternatively, a selection is performed for spontaneously occurring mutants.

By the term “a probiotic product” is meant any product which comprises a probiotic bacterium. A probiotic product comprising a strain according to the invention may be administered in the form of a food product or a dietary supplement. The Bifidobacterium longum may, for example, be incorporated in a dairy product, such as milk, and in particular a fermented dairy product, optionally in combination with other lactic acid bacteria, for example with yogurt ferments, or in other food products such as a snack bar, or beverages such as juice.

The probiotic product comprising Bifidobacterium longum can also be provided as a dietary supplement in the form of a powder, tablet, such as a lozenge or effervescent tablet, pastille, capsule, chewing gum, in individual sachets or as a component of a more general composition such as oil drops, an emulsion or a paste, or in any other suitable carrier determined by those of skill in the art to be an effective carrier for live microorganisms.

Probiotic bacteria are live microorganisms, and this can be a challenge during formulation and storage of probiotic products. Probiotic bacteria are especially sensitive towards temperature, moisture content, and oxygen and other ingredients in a formulation matrix. It is preferred that the bacteria of the invention remain viable after prolonged storage in order for the bacteria to impart their beneficial effect upon administration of the probiotic product of the invention to the individual in need thereof.

By the term “viable” is meant that the cell is alive and capable of forming a colony in a petri dish during pour plating or spread plating. The number of viable probiotic bacteria is determined as the number of colony forming units (CFU) by pour plate or spread plate methods with incubation under conditions suitable for growth of the probiotic strain(s). By this method cells capable of growing and forming colonies will be counted. When a number is given in the present specification and claims, it should be understood as CFU/mL unless the context indicates otherwise. In some embodiments, the probiotic product of the present invention comprises at least 10⁹ CFU/unit at end of shelf life (EOS). The end of shelf life may be at least 3 months, such as at least 6 months, at least 9 months, at least 12 months, at least 18 months, or at least 24 months.

The probiotic bacteria to be used in the probiotic products of the invention are generally frozen or freeze-dried. In order to obtain a high viability the bacteria are mixed with a cryoprotectant before they are frozen or freeze-dried.

The term “a cryoprotectant” denotes a substance that is able to improve the survival during freezing and/or drying and to improve the storage stability of bacteria. The cryoprotectant used herein generally comprises a saccharide or sugar alcohol such as glycerol.

The saccharide may be a mono-, di-, oligo- or polysaccharide, or a mixture of at least two saccharides. The composition may even comprise three, four or more saccharides. In some embodiments, the composition comprises a mixture of at least one mono- or disaccharide and at least one oligosaccharide. In other embodiments, the composition comprises a mixture of at least one mono- or disaccharide and at least one polysaccharide.

Monosaccharides useful in the probiotic product of the present invention include glucose (also known as dextrose), fructose, ribose and galactose. Disaccharides useful in the probiotic product of the present invention include among other sucrose, trehalose, maltose and lactose. The composition may comprise one or more mono- or disaccharides, such as one, two or three or even more different saccharides.

In some embodiments the probiotic product of the invention comprises at least one oligosaccharide. An oligosaccharide is a saccharide polymer containing three to nine monosaccharides. Fructo-oligosaccharides (FOS), which are found in many vegetables, consist of short chains of fructose molecules. Galactooligosaccharides (GOS), which also occur naturally, consist of short chains of galactose molecules. These compounds can be only partially digested by humans. The composition may comprise one, two or even more different oligosaccharides.

In some embodiments the probiotic product of the invention comprises at least one polysaccharide. Polysaccharides are polymeric carbohydrate molecules composed of more than ten monosaccharide units bound together by glycosidic linkages and on hydrolysis give the constituent monosaccharides or oligosaccharides. They range in structure from linear to highly branched. Examples of polysaccharides to be used in a probiotic product of the invention are maltodextrin, cyclodextrin, alginate, pectin, chitosan, starch and inulin. The composition may comprise one, two, three or even more different polysaccharides.

As an example, the cryoprotectant may comprise a mixture of a disaccharide, such as sucrose or glucose, and a polysaccharide, such as maltodextrin.

The addition of oligo- or polysaccharides such as FOS, GOS, inulin and other polysaccharides can assist in reduction of the water activity and has the further advantage that oligo- and polysaccharides are not quite as sweet as mono- and disaccharides and further that they add fibers to the composition.

Polyols (sugar alcohols) have the general formula HOCH₂(CHOH)ₙCH₂OH. They are commonly added to foods because of their lower caloric content and less sweetness than sugars. Furthermore, they are not broken down by bacteria in the mouth or metabolized to acids, and thus do not contribute to tooth decay,

The composition may further comprise at least one polyol such as erythriol, inositol, isomalt, mannitol, maltitol, sorbitol, or xylitol, or a mixture thereof. Preferred polyols are xylitol, sorbitol and mannitol. The composition may comprise one, two, three or even more different polyols.

The cryoprotectant may further comprise a peptide, protein, protein hydrolysate or a mixture thereof. Examples of peptides and proteins to be used herein are casein, pea, whey, albumin, soy protein, glutamic acid or gelatin, and any isolate or hydrolysate thereof. Other additives, e.g. antioxidants such as ascorbate, sodium citrate, propyl gallate may also be present.

The present invention also relates to a probiotic comprising an isolated strain according to the invention and a cryoprotectant, such as a saccharide.

Combinations of several species or strains of probiotic bacteria can be used, i.e. 2, 3, 5, 6, 7, 8, 9, 10, 11, 12, 13, 14, 15, 16, 17, 18, 19, 20, 21, 22, 23, 24, 25 or even more of the species and strains listed herein. In presently preferred embodiments, only one, two, three, four or five different strains are present in a probiotic product according to the invention.

In addition to the probiotic bacteria, one or more other active ingredients, for example one, two, three, four or more active ingredients selected from the group consisting of vitamins such as vitamin A, D, E, K2, C, B2, B6, B12, biotin, niacin, folic acid; minerals such as zinc, selenium, chromium, copper, calcium, chloride; and vegetable extracts such as cranberry extract/juice, royal jelly could be included in the probiotic product.

It is contemplated that in order to obtain a therapeutical effect, the probiotic product should be administered daily for at least one week, and advantageously for a longer period such as at least 2 weeks, at least 4 weeks, at least 6 weeks, at least 9 weeks, preferably at least 12 weeks, in an amount corresponding to at least 10⁶ CFU, such as at least 10⁷ CFU, preferably at least 10⁸ CFU, generally between 10⁹ CFU and 10¹² CFU of Bifidobacterium longum.

In the present studies the probiotic product comprises Bifidobacterium longum as the active ingredient. Bifidobacterium longum may be used as the only active ingredient. Alternatively, the probiotic product as described herein may comprise further compounds of interest such as other bacterial strains, vitamins, prebiotics, fibers or other compounds which may have a beneficial health effect.

The other bacterium may be selected from the group consisting of Bifidobacterium lactis, Lactobacillus rhamnosus, Lactococcus lactis subsp. lactis, Lactococcus lactis subsp. cremoris, Leuconostoc lactis, Leuconostoc mesenteroides subsp. cremoris, Pediococcus pentosaceus, Lactococcus lactis subsp. lactis biovar. diacetylactis, Lactobacillus casei subsp. casei, Streptococcus thermophilus, Bifidobacterium longum, Lactobacillus lactis, Lactobacillus helveticus, Lactobacillus fermentum, Lactobacillus salivarius, Lactobacillus delbrueckii subsp. bulgaricus and Lactobacillus acidophilus.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### BRIEF DESCRIPTION OF THE FIGURES

FIG. 1 are graphs showing growth and viability of BB-12® and BB-46.

FIG. 2 are graphs showing the fatty acid profile of BB-12® in CDM-Ref and CDM-no-Tween, of BB-46 in CDM-Ref as well as of the FAS-mutant of BB-46 in CDM-no-Tween.

### DEPOSIT AND EXPERT SOLUTION

The applicant requests that a sample of the deposited microorganisms stated below may only be made available to an expert, subject to available provisions governed by Industrial Property Offices of States Party to the Budapest Treaty, until the date on which the patent is granted.

TABLE 1
The applicant has made the following deposits at
a Depositary institution having acquired the status of
international depositary authority under the Budapest Treaty
on the International Recognition of the Deposit of Microorganisms
for the Purposes of Patent Procedure: Leibniz Institute DSMZ-
German Collection of Microorganisms and Cell Cultures
Inhoffenstr. 7B, 38124 Braunschweig, Germany.

| Strain | Accession No. | Deposit date |
|---|---|---|
| B. animalis subsp. lactis BB-12 ® | DSM 15954 | 2003 Sep. 30 |
| B. longum subsp. longum BB-46 | DSM 15955 | 2003 Sep. 30 |

### SEQUENCE LISTING

The nucleotide and protein sequences of the multifunctional microbial type I fatty acid synthase gene in BB-46 (BL/wild type) and in the mutant of BL is given in Table 2.

TABLE 2
Sequence listing

| SEQ | | |
|---|---|---|
| ID | | |
| NO. | Comment | Sequence |
| 1 | Mutated DNA | ATGACCACCTTCTTCCTCAATCGCCTAGCCACCGAGCCCCATG |
| | | CGCTCGCCTTCGCCGGCCAGTCCACCCCGTGGCCGGTAGCCC |
| | | TGGCAGACCAGACCACCGATCCCAGCCTTGACGAGGCCCTGC |
| | | GCGGCCACATCGCCGCATCCAACCGACTGCTCACCCCGGTCG |
| | | CCGCCGACCTGCTGACCACCACCGGCCGTCCGGTCGACCTGT |
| | | TCGGCTTCACTCCGAACCCGGCCCGCCTCGGCGCCGCAGCCG |
| | | ACGCCACCGCGTCCGTGGAAGGCGTGGCACTGACCCAGCTC |
| | | GGCGCCCTGCTCGACCTCGAACACCTCGGCTACAGCGTGGCC |
| | | CAGGCCAAGCCGCTCGCCGTGCTCGGCCACTCCCAGGGCGT |
| | | GCTCGCCGTGCACATGGCCCGCGCCATCGAGGCCGCCGGCT |
| | | CCATCGAGGCCGCCGGCAAGACCCTTGACGAGATTCTGGCCG |
| | | TCGCCGCCCTGATCGGTGCAGCCGGCACTCGCCGCGTGCGC |
| | | GAACTGGGCCTGAACCCCAAGTATGGCGAAGCCAGTCCGATG |
| | | CTCTCCGTCAAGGGCGCCACCCGCGAACAGGTTGAGGCTCTG |
| | | GTGGCCCGCGTCAACAACGCTCGCGGCCCGATCTCCATCGCC |
| | | GTCACCAACTCCGACAACCACCACGTGCTTTCCGGCTACCCG |
| | | GAGGACCTGTCTGCCTTCGCCCTCGAAGCCGAGCGCGAACAC |
| | | CAGCATCAGGCCAAGCTGCGCGAGCAGAAGCTCCACGGCGG |
| | | CACCGTGTTCAACCCGACCCTCGAATACCTTGAGGTCACCCTG |
| | | CCCTTCCACTCGCCGCTCATGGCCGACGCCGTCGAGCAGACC |
| | | GTGGCCTGGGCCGGCGCCTGCGGCTTCGACCAGAAGCGCAC |
| | | CCGTGCGCTTGCCGAGGAAGTGCTTCTGAACCATGTGGACTG |
| | | GAACGCCCGCGTCAAGGCGCTGTTCGACGCCGCCGACCCGTC |
| | | CAAGCTGTGGATCGTGGACCTCGGCCCCGGCAACACGCTCGG |
| | | CAAGCTGATCGGCAATGTGGTGCAAGGCACCGGCATCGGCGT |
| | | GGTCGAAGCCACCACGCTGGCCGAGCGCTCCACGCTCTCCAC |
| | | GCTGGAAAGCGAGCCTGAGCGCACCCAGAACTGGAAGGCATT |
| | | CGCCCCGCGCGTCATCAACACCCCGGCCGGCGCCAAGCTGGT |
| | | CACCAAGTTCTCCAAGCTCACCGGCAAGCCGCCGGTACTGTT |
| | | GCCCAGCATGACCCCCACCACCGTGGAACCGGAAATCGTGGC |
| | | CGCCGCGGCCAACGCCGGCTACTGGGCCGAGCTCGCCGGCG |
| | | GCGGCCAGGTGACCGCCGAAGTGTTCGACCGCCACATCGCCA |
| | | CCCTCGAAGACGAGCTGGAGGAAGGCCGCACGGTCGAATTCA |
| | | ACGCGATGTTCATGGACCGTTACCTGTGGAACCTGCAGTTCG |
| | | GCTCCTCCCGCATCGTGCCCAAGAAGCGCGCATCCGGCGCGC |
| | | CGATCGACGGCGTGGTCGTCTCCGCCGGCATCCCCGAGCTG |
| | | GACGAAGCCGTGGCCCTGATCAAGTCGCTGCAGGCGGACGG |
| | | CCTGCCGTACGTGAGCTTCAAGCCCGGCACCGTGGACCAGAT |
| | | TCGTCAGGTCGTGCGCATCGCCAAGGCCGTGGCCCCCACCAC |
| | | CATCATGGTTCAGGTCGAGGGCGGTGAAGCCGGCGGCCACC |
| | | ACAGCTGGGAAGCGCTGGATGACCTGCTTGCCGCCACCTACG |
| | | CCGAAGTGCGCGCCTGTGACAACCTCGTGCTGGTCGCCGGCG |
| | | GCGGCATCGGCACTCCCGAACGCGCGGCCGACTACATTTCCG |
| | | GCCAGTGGGCCCACGCCTACGGCCTGCCCGACATGCCGGTC |
| | | GACGGCGTGCTTATCGGTACCGCCGCGATGACCGCCAAGGA |
| | | GGCTCACACCAGCCCCGAAGTCAAGCAGCTGCTCGTCAAGAC |
| | | GCCCGGTATTCCGGCCGACGCCAAGTCCGACGACGTGTTCGC |
| | | CCCGCAGGCCGCCCACTGGGTGCCGTCCGGCAAGTCCGTCG |
| | | GTGGCATGTCCTCCGGCCTGAGCCACCTGCACGCCGACATTT |
| | | ACGAGCTCGAGAACGACTCCGCCGAATGCGGCCGTCTGCTCG |
| | | TGCGCGTCATGAAGCACCCCGAAGAGCTCGACTCCCGCCGCA |
| | | AGGAAATCATCGAAGCCCTGAACAAGACCGCCAAGCCTTACTT |
| | | CGGCGACCTGGCCTCCATGACCTACCTTGAGTGGGCGCAGCG |
| | | CTTCGCCGAACTCGCCTTCCCGTGGGTCGACCCGACTTACGC |
| | | CGACCGCTTCCAGCACCTGCTGCAGCGCATCGAAGCCCGTGT |
| | | GAACGACACGGACTCCGGCGAATTCACCTCCAAGTTGTTCGC |
| | | CGCTGATGGCGTTTCTGCTGAAGAGGCAGCTGCCGCTGGCCT |
| | | GCTGACCCACGACGACATTCTCGCCGACCCGGCCCCCGCGCT |
| | | CGAAAAGCTCGCACTGGCCTACCCGCAGACCGCCGAACTCAA |
| | | GGTGGTCCCGACCGACGTGGCTTGGTTCCCGGTGCTCGTGCG |
| | | CGAATACCCCAAGCCCATGCCGTTCGTGCCCGTCATCGACAA |
| | | CGACCTGCTGCGTTGGTGGGGGCAGGACCAGCTCTGGCAGT |
| | | CCGAAGACCAGCGCTACTCCGCCGATTCCGTGCGTGCCATCC |
| | | CCGGCCCGATCTCCGTTGCCGGCATCACCACCATCGACGAGC |
| | | CGATCGCCGACATCCTCGGCCGCTTCGAAACCGCCGCCATCA |
| | | AGCGCGTGCAGGACGAACAGCAGGCCGCCGATGCCGCTGAG |
| | | AACGACGATTTCGCCGCACTTGGCGAGGCCACCAGCGCCGAA |
| | | GACTTCATCCGCAAGTCCCCGAACATCTCCTGGGTCGGCCAC |
| | | ATCACCGACAACCCGGCCTACGGCACCGCCCTGGGCGACCAG |
| | | TACTACGAGATCCGCGCGTTTGACGCCGCGGCCGGCAAGTAC |
| | | GATCTGGACATCCACCTCGACACCTACTGGGACAACGACCCG |
| | | GACGGCGGCACCTCCAAGCACGCCGTGCGCGATATCGTCATC |
| | | CCGCTCATCGTGGAAGGCGCCGAGCCCGGCCGTGTGCCGGT |
| | | CGTGGACCGCGAACGCCTGATTCCGGACGTCTACGCGATGCT |
| | | CGCCGCCACCGCAGGCATCGGCAACACCGCCATCACCGGCG |
| | | ACAAGCTCACCGAGATGCCGCAGCTTGACGGCAAGACCTCTG |
| | | ACGAGCGTCCGTTTGCTACCGCCACCGCCGCCTACACCCTCT |
| | | CCGCCAACCTCGGCTTCGACCACGAAGCGGCCACCGGCGCC |
| | | GCTCTGCCGACGACCCTGCAGCCCAGCCGCATCGCTCCCGAT |
| | | GCGCTGGTCGGCCCGGCCTGGCCGGCGATTTACACGGCGCT |
| | | CGGCTCCGTGTACGTCAAGGGCTACCCGGTGATTGAAGGCCT |
| | | GCTCAACGCCGTTCACCTCGACCACCTCATCGAACTTGAGGTG |
| | | AACGAGGAGGACCTGCTTAAGCACACCGGTGAAACCATCACC |
| | | CTGAAGTCTTGGGCTGAGGATTACTTCGAGTCCGCCTCCGGC |
| | | CGCGTGGTCACCATCCACGTGACCCACACCGCTGCCGACGGC |
| | | ACGCTGCTTGCCCGCGAAACCGAGCGTTTCGCGATTCGCGGC |
| | | CGCGTCTACTCCGATGCTTTGCCGGCAGACGCCCCCGAGTTC |
| | | GGCGACGCCGAGCACGAGGAGATCGCGCCCACGCCGCGCCG |
| | | TCTGCTGCGCCGCGTGAAGGTCGTCGCCCCACACGACATGAC |
| | | CGCCTTCGCCCGCACTTCCGGCGACTTCAACCCGATCCACAC |
| | | CTCCACTCGTGGCGCTCGCATCTCCGGTTTGGCCGCTCCGCT |
| | | TGTGCACGGCATGTGGCTGTCCGCCACCGCCCAGCACGCCGT |
| | | GCAGGCGCTGGACGAGAAGGGTGCCCACTACGAGATTGCCG |
| | | GCTGGACCTACAACATGTACGGCATGGTTCAGCTTGACGACA |
| | | CCGTCGAAATCTCCGTCGAGCGCGTCGGCAAGGTGCGCCACG |
| | | GCGGCATGACCCTCGAAGTCACCTGCCGTATCGACGGCCAGC |
| | | TCGTCTCCCGCGGCACCGCACTGGTCCGCGCGCCGCGCGCC |
| | | GCCTTCGTCTACCCCGGCCAGGGCATCCAGAAGCAGGGCATG |
| | | GTTCTGGACGAGCGTGCCAAGTCCGCCGCCGCCCGCGACGT |
| | | GTGGGAGCGCGCCGACAAGCTCACCCGCTCCAAGCTCGGCTT |
| | | CTCGATCCTGGGCCTCGTGCGCGATAACCCGAAGGAGCTCAC |
| | | CGCCAACGGCGTGACCTATCGCCACCCCGAAGGCCTGCTCAA |
| | | CCTGACGCAGTTCACGCAGGTGGCGCTCGCCACCGTGGCCTT |
| | | CGCCCAGACCGCTCGTCTGCGCGAAGCCGGCGCCGACATCTG |
| | | GCCCGCCTACTTCGCCGGCCACTCGCTCGGCGAATACAACGC |
| | | GCTGTCCGCGTTCGCCGACGTGATTCCGCTGGAAACCGTGCT |
| | | GGAGCTCGTGTTCCACCGTGGTTCCACCATGCACCACCTCATC |
| | | GAGCGTGACGCACAGGGTCGCTCCAACTACCGCATGGGTGCC |
| | | CTGCGCCCGAACCAGTTCGGTGTGGACGACGCGCACGTCAAG |
| | | GAATACGTGGAATCCGTGGCCAAGGCGTCCGGCGAATTCCTG |
| | | GAGATCGTGAACTACAACCTCGCCGGCCAGCAGTACGCCATC |
| | | GCCGGTACGATCGCCGGACTCAAGGCTCTGAAGGCCGATTCC |
| | | GCCCGCCGCGTGGCCGCATTCGGTGGCAAGCCCGCGTTCAT |
| | | GCTGGTGCCCGGCATCGATGTGCCGTTCCACTCCACACTGCT |
| | | GCGCAAGGGCGTGCCGGAATTCCGCGACAAGCTCGACGCGC |
| | | TGCTTCCCGCTTACATCGACTACCGTGGCCGTCTGGTGGACC |
| | | GCTACATCCCGAACCTGGTGGCCACCCCGTTCGAGATGACCA |
| | | AGGAATTCGCCGCCAAGATCCTCGAAGTCGTGCCGTCCGAGC |
| | | GTATCAAGGCCGTGCTCGACGACCCGGCCGTGTGGGATTCCT |
| | | ACGCCGATGACGACCAGAAGCTCGGCCGTCTGCTCTTGACCG |
| | | AACTGCTCTCCTGGCAGTTCGCCTCCCCGGTGCGTTGGATTG |
| | | AAACCCAGGCGCTGCTCTTCGGTCAGCGCGAACAGGGCGGC |
| | | CTCGGCGTGGAGGAATACGTCGAAGTCGGCCTCGGCAACGC |
| | | CCCGACCCTGGCCAACCTCGGTGCCAAGACCCTGCGTCTACC |
| | | CGAGTTCGCCGGCAACGATACGGTCGTCTACAACGTCGGCCG |
| | | CGATGAAGGCCGCGTCTACATGACCGACACCGATTCGCTGGT |
| | | TCCCGACGATGAGCCGGAAGAGGCTGCGGCCTCGGCCCCGG |
| | | TCGAAACCGCTCCTGCCGGTGGTTCCGCCGTCGCCAAGCTCG |
| | | GTTCCGGTGCCGCTCCGGCAAATGCCGTGACTGAGGCTGCTC |
| | | AGACAACGGATGGCTCCGTCGCCGGACAGTCCACTGGACTGT |
| | | CCGACTCCTCGCGCGGCCTGCAATCAGGGCAGGCCGCGGCC |
| | | GGCGCACCGTCCGGTGCCGCCGTCGCCGACCTGCCCTTCAAG |
| | | GCTTCCGATGCCATCGGCGTGCTCATGGCCTACTCCGCCAAG |
| | | GTGCGCATCGACCAGATCGGCAGCAACGACACCACTGACACC |
| | | CTGACCAACGGTGTCTCTTCTCGCCGCAACCAGCTGCTCATG |
| | | GACATCAGCTCCGAGCTGGGAGTCGCCAGCGTGGACGGTGC |
| | | TGCTGAAGCGACGCTCGACAAGCTCGCCCAAATCGTCAACAA |
| | | GGCCGCCCCGAACTACAAGCCGTTCGGTGCCGTGCTCTCCGA |
| | | GGCGCTGCGCGACCGCCTGCGCTCGCTGTTCGGTGCCGCCG |
| | | GCGTCAAGCAGCAGTACATCCGCGACCGCGTGACCAACGTAT |
| | | GGCAGCTCGGTGAAGGTTGGGTGGCCAGCGTGCTCGCCGCA |
| | | CTGCTGCTCGACACTCGCGAGGGTGCCAGCTCCCGTGGCGG |
| | | CGACCTGGCCAAGCTGCCCACCGCCGCCGTGCAGAACAAGCC |
| | | GGAAGCCGACAAGCTGATTGATGCGGCCGTTGAGGTCGTCGC |
| | | CCAGCTCAAGGGTGTTGCCGTGGCCCTGCCGTCCGCCGGTG |
| | | GTGCCGCTGGTGGCGCTGTGGTCGATTCCGCCGCGCTCGAC |
| | | GCCTTCGCCGAGAAGGTCACCGGTTCCAACGGCGTGCTCGCC |
| | | GCCACCGCCCGCTTCGTGCTGAACGAGCTCGGCGTGGCCGCT |
| | | CCGGCTCCGGAAGAATCCGAGGACGAGAACGCCGCCGTCGT |
| | | GGCCGCGGTTGAAGCCGAACTTGGCTCCGACTGGCCTAAGCA |
| | | GGTCGCTCCGCGCTTCGACGCCAACAAGGCCATCCTCTTCGA |
| | | TGATCGGTGGGCCTCCGCCCGTGAAGACCTCGCCCGCGCCTA |
| | | CTACGACAACGATCCCGCCGCCTTGAACGGCAGCTTCATCGG |
| | | CCTTGGCAAGACCATCGCCGCCGAAGCCCAGTGGTTCGCCAA |
| | | CGAGTCGGAGAGCGAAGAACTCAAGGCCGCCTTCCAGAAGG |
| | | CCGGCTCCGAGGCCCTCGAACAGGTTGCGTCCAACAAGAACG |
| | | CCTCCCGTTACGCGAATGATATCGCCATCGTCACCGGCGTGA |
| | | GCCCGAACTCGATTGCCGCTCAGGTGGTCGAAGGCCTGCTCG |
| | | CAGGCGGTGCCACTGTGGTGGCCACCTCGCACAGCTTCAAGC |
| | | CGTCCATTAAGGCTTGGGCTAAGCAGGCCTACCGCGAGCACG |
| | | CTACCGGCAACGCCAAGCTGTGGCTGGTTCCGGCCAACCTCT |
| | | CCAGCTACCGCGACGTCGACGCACTGGTCGACTGGGTGGGC |
| | | CACGAGCAGAAGAAGACCTCCGGTGCTACCACCACGATTCTG |
| | | AAGCCCGCGTGGGAGCCGACTCTGTTCTTCCCGTTCGCCGCT |
| | | CCGCCGGTCCATGGCACGCTCGCCGACTCCGGCGACCTGTTC |
| | | GAATCGCAGGCCCGACTGATGCTGTGGGGCGTGGAACGCGC |
| | | CATCGCCGGCTTCAGCCACATCGGTGCCGACACCAACGTGCA |
| | | GCACAAGCTCCACGTGGTGCTGCCCGGTTCCCCGAACCGCGG |
| | | CGTATTCGGCGGCGACGGTGCCTACGGCGAAGTCAAGAGCG |
| | | CCTTTGACGCCATCGTCAACCGTGCCCGCGCTGAAAAGGTGT |
| | | GGTCGAGCCGCGTCACCTTCGCTCACCCGAAGATCGGCTGGG |
| | | TGCGTGGCACCGGCCTGATGGTCGGTAACGACCCGCTGGTCG |
| | | CCGTCGTTGAGCGTCACGGCATCCGCACCTACTCCACCGCAC |
| | | AGATTGCGGCCAAGCTGCTCGACCTGTGCACTGCCGAATCGC |
| | | GCGAGCAGGCCCTCAAGGCCCCGCTCGATGTGGACCTGACC |
| | | GGTGGCCTCGGCTCCGAGCCCATCGACATCAAGGCCCTGCGT |
| | | GCGGAGGCCATGGCCGACGCCGAGAAGGAAGCTGCTGCGGC |
| | | CTCGTCTCAGGAAACGGATGGCTCCGTCGCCGGAAAGTCCAC |
| | | TGGACTGTCCGACTCCTCGCGTGGGCAGCAAATCAAGGCGCT |
| | | GCCCACACCGATCGTCACCAAGCAGGCTCCGGTCGACTTGAA |
| | | CGATTGGACCAACGTCACTGCCAAGCCGGAAGACGAAATCGT |
| | | CATCGTTTCCGTCGGTGAGCTGGGCCCGTGGGGCTCCGGTCG |
| | | TACGCGCGCCCAGGCCGAGCTCGGCATCCACTCGGACGGCA |
| | | CCGTCGACCTCAGCGCCGGCGCAGTGCTCGAACTCGCGTGGA |
| | | ACATGGGCCTGCTGACTTGGGCCGACAGCCCGAAGCCCGGCT |
| | | GGTACGACACCGACGGCAACCTCGTGCCCGAAGAGGACATCG |
| | | CCGAGCGCTACCACGACGAGGTCGTGGCCCGCTCCGGCATCC |
| | | GCCCGTTCGAGGAAGGCATGGGCAACGACTACAAGGACGGT |
| | | GCTGACGAAGAGGAAGCCGAGGTCTTCCTCGACCACGACGTC |
| | | ACCTTCTCTGTGCCCACCCGCGAAGTCGCCGCCGAATACGTC |
| | | AAGTTGGACGAGGCTCACACCACCATCGCCCCGGATGAGGAA |
| | | TCCGGCGAATGGAACGTGACCCGCCACGCCGGCTCGATGATC |
| | | CGCGTGCCGCGCCGCGCCACCATGACCCGCACAGTCGGCGG |
| | | CCAGTTCCCGAAGGGCTTCGACCCGACCCGATGGGGCATTCC |
| | | CGCCTCGATGGTCGGCGACGTCGACAAGATCGCCCTGTGGAA |
| | | CATCGTCACCACTGTGGACGCCTACCTGGGCGCCGGCTTCAC |
| | | CCCGGCCGAAATCCTCGAGTCCATCCACCCCTCGCTGGTGGC |
| | | TTCGACCCAGGGCACCGGCTTCGGTGGCATGATGTCGATGCG |
| | | CAAGCTGTACCTCGACCGCTTCCTCAACCACGAGATCCCGACC |
| | | GACATCCTGCAGGAAGCCCTGCCGAACGTGGTCGCGGCTCAC |
| | | GTGATGCAGTCCTACATCGGCGGCTACGGCAACATGATTCAG |
| | | CCGGTCTCCGCCTGCGCCACCGCAGCCGTCTCCCTTGAAGAG |
| | | GGCGTCGACAAGATCGCCCTCGGCAAGGCCGACTTCGTGGTC |
| | | ACCGGTGCGATCGACGACATCGGCGTGGAATCCGTGATCGGT |
| | | TTCGGCAACATGAACGCCACCGCCAACTCGGAGGAAATGTAT |
| | | GGCAAGGGTATCGACGCGCGCTTCTTCAGCCGCGCGAACGAT |
| | | CGTCGCCGTGGCGGCTTCCTGGAATCCCAGGGCGGCGGCAC |
| | | GATCCTCGTGACCCGCGGTGACATCGCCGAAAAGCTCGGCCT |
| | | GCCGGTTGCGGCCGTGGTCGGCTTCATCCACTCCTACGCGGA |
| | | TGGCGCTCACACCTCGATCCCGGCTCCGGGCCTCGGTGCCCT |
| | | GGCTGCCGGCCTCGGCGGCAAGGACTCCAAGCTCGTGCACG |
| | | ACCTGGCTAAGCTCGGCGTCTCAGCCGACGACATCGCCGTGG |
| | | TCTCCAAGCACGACACGTCCACCAACGCCAACGACCCGAACG |
| | | AATCCGAGCTGCACAACACGCTGGCCCACGCCATCGGCCGCA |
| | | CCGACGGCAACCCGCTGTTCGTGATCTCGCAGAAGACGCTTA |
| | | CCGGCCACGCCAAGGGTGGTGCCTGCATCTTCCAGGTCAACG |
| | | GTCTGACGCAGCTGTTCAAGTCCGGCGTGATTCCGGCGAACG |
| | | CGGCGCTCGACTGCGTGGATCCGAAGCTCCAGCGCGACGAC |
| | | CACATGGTCTGGGTTAGGAAGCCCCTGCGCATCGGCGGTGGT |
| | | GAGGACGAGTTCGGTCGCGAGACCGCTGGTCGCCCGGTCAA |
| | | GGCCGGTCTGGCCACCTCGCTCGGCTTCGGACATGTTTCCGG |
| | | CTTCGTGGCTCTGGTCCATCCGGGTGCCTTCGAGGCGGCCGT |
| | | GGCCAAGGCCGATGGTGAGGCCGCACTGGAAGCTTGGCGTG |
| | | AGCGTGCCAACGCCCGCCTGGCTGCCGGCCAGCGTCACCTTG |
| | | AGGAAGGCATGATGGGTCGCGCCGCGCTCTACGAGCCGATC |
| | | GACAACCGTCGCTTCCGCGAGGATCACCGTGGCTACGACCAC |
| | | CACGAGGTCGAAAAGGCGATGCTCCTCAACCCGGACGCCCGC |
| | | CTCGGTGCCGACGGCTACTACGAGGCCTGA |
| 2 | Mutated | MTTFFLNRLATEPHALAFAGQSTPWPVALADQTTDPSLDEALRG |
| | protein | HIAASNRLLTPVAADLLTTTGRPVDLFGFTPNPARLGAAADATAS |
| | | VEGVALTQLGALLDLEHLGYSVAQAKPLAVLGHSQGVLAVHMA |
| | | RAIEAAGSIEAAGKTLDEILAVAALIGAAGTRRVRELGLNPKYGE |
| | | ASPMLSVKGATREQVEALVARVNNARGPISIAVTNSDNHHVLSG |
| | | YPEDLSAFALEAEREHQHQAKLREQKLHGGTVFNPTLEYLEVTLP |
| | | FHSPLMADAVEQTVAWAGACGFDQKRTRALAEEVLLNHVDWN |
| | | ARVKALFDAADPSKLWIVDLGPGNTLGKLIGNVVQGTGIGVVEA |
| | | TTLAERSTLSTLESEPERTQNWKAFAPRVINTPAGAKLVTKFSKL |
| | | TGKPPVLLPSMTPTTVEPEIVAAAANAGYWAELAGGGQVTAEVF |
| | | DRHIATLEDELEEGRTVEFNAMFMDRYLWNLQFGSSRIVPKKRA |
| | | SGAPIDGVVVSAGIPELDEAVALIKSLQADGLPYVSFKPGTVDQI |
| | | RQVVRIAKAVAPTTIMVQVEGGEAGGHHSWEALDDLLAATYAE |
| | | VRACDNLVLVAGGGIGTPERAADYISGQWAHAYGLPDMPVDGV |
| | | LIGTAAMTAKEAHTSPEVKQLLVKTPGIPADAKSDDVFAPQAAH |
| | | WVPSGKSVGGMSSGLSHLHADIYELENDSAECGRLLVRVMKHP |
| | | EELDSRRKEIIEALNKTAKPYFGDLASMTYLEWAQRFAELAFPWV |
| | | DPTYADRFQHLLQRIEARVNDTDSGEFTSKLFAADGVSAEEAAA |
| | | AGLLTHDDILADPAPALEKLALAYPQTAELKVVPTDVAWFPVLVR |
| | | EYPKPMPFVPVIDNDLLRWWGQDQLWQSEDQRYSADSVRAIP |
| | | GPISVAGITTIDEPIADILGRFETAAIKRVQDEQQAADAAENDDF |
| | | AALGEATSAEDFIRKSPNISWVGHITDNPAYGTALGDQYYEIRAF |
| | | DAAAGKYDLDIHLDTYWDNDPDGGTSKHAVRDIVIPLIVEGAEP |
| | | GRVPVVDRERLIPDVYAMLAATAGIGNTAITGDKLTEMPQLDGK |
| | | TSDERPFATATAAYTLSANLGFDHEAATGAALPTTLQPSRIAPDA |
| | | LVGPAWPAIYTALGSVYVKGYPVIEGLLNAVHLDHLIELEVNEED |
| | | LLKHTGETITLKSWAEDYFESASGRVVTIHVTHTAADGTLLARET |
| | | ERFAIRGRVYSDALPADAPEFGDAEHEEIAPTPRRLLRRVKVVAP |
| | | HDMTAFARTSGDFNPIHTSTRGARISGLAAPLVHGMWLSATAQ |
| | | HAVQALDEKGAHYEIAGWTYNMYGMVQLDDTVEISVERVGKVR |
| | | HGGMTLEVTCRIDGQLVSRGTALVRAPRAAFVYPGQGIQKQGM |
| | | VLDERAKSAAARDVWERADKLTRSKLGFSILGLVRDNPKELTAN |
| | | GVTYRHPEGLLNLTQFTQVALATVAFAQTARLREAGADIWPAYF |
| | | AGHSLGEYNALSAFADVIPLETVLELVFHRGSTMHHLIERDAQG |
| | | RSNYRMGALRPNQFGVDDAHVKEYVESVAKASGEFLEIVNYNLA |
| | | GQQYAIAGTIAGLKALKADSARRVAAFGGKPAFMLVPGIDVPFH |
| | | STLLRKGVPEFRDKLDALLPAYIDYRGRLVDRYIPNLVATPFEMTK |
| | | EFAAKILEVVPSERIKAVLDDPAVWDSYADDDQKLGRLLLTELLS |
| | | WQFASPVRWIETQALLFGQREQGGLGVEEYVEVGLGNAPTLAN |
| | | LGAKTLRLPEFAGNDTVVYNVGRDEGRVYMTDTDSLVPDDEPEE |
| | | AAASAPVETAPAGGSAVAKLGSGAAPANAVTEAAQTTDGSVAG |
| | | QSTGLSDSSRGLQSGQAAAGAPSGAAVADLPFKASDAIGVLMA |
| | | YSAKVRIDQIGSNDTTDTLTNGVSSRRNQLLMDISSELGVASVD |
| | | GAAEATLDKLAQIVNKAAPNYKPFGAVLSEALRDRLRSLFGAAG |
| | | VKQQYIRDRVTNVWQLGEGWVASVLAALLLDTREGASSRGGDL |
| | | AKLPTAAVQNKPEADKLIDAAVEVVAQLKGVAVALPSAGGAAGG |
| | | AVVDSAALDAFAEKVTGSNGVLAATARFVLNELGVAAPAPEESE |
| | | DENAAVVAAVEAELGSDWPKQVAPRFDANKAILFDDRWASARE |
| | | DLARAYYDNDPAALNGSFIGLGKTIAAEAQWFANESESEELKAA |
| | | FQKAGSEALEQVASNKNASRYANDIAIVTGVSPNSIAAQVVEGL |
| | | LAGGATVVATSHSFKPSIKAWAKQAYREHATGNAKLWLVPANL |
| | | SSYRDVDALVDWVGHEQKKTSGATTTILKPAWEPTLFFPFAAPP |
| | | VHGTLADSGDLFESQARLMLWGVERAIAGFSHIGADTNVQHKL |
| | | HVVLPGSPNRGVFGGDGAYGEVKSAFDAIVNRARAEKVWSSRV |
| | | TFAHPKIGWVRGTGLMVGNDPLVAVVERHGIRTYSTAQIAAKLL |
| | | DLCTAESREQALKAPLDVDLTGGLGSEPIDIKALRAEAMADAEK |
| | | EAAAASSQETDGSVAGKSTGLSDSSRGQQIKALPTPIVTKQAPV |
| | | DLNDWTNVTAKPEDEIVIVSVGELGPWGSGRTRAQAELGIHSD |
| | | GTVDLSAGAVLELAWNMGLLTWADSPKPGWYDTDGNLVPEEDI |
| | | AERYHDEVVARSGIRPFEEGMGNDYKDGADEEEAEVFLDHDVT |
| | | FSVPTREVAAEYVKLDEAHTTIAPDEESGEWNVTRHAGSMIRVP |
| | | RRATMTRTVGGQFPKGFDPTRWGIPASMVGDVDKIALWNIVTT |
| | | VDAYLGAGFTPAEILESIHPSLVASTQGTGFGGMMSMRKLYLDR |
| | | FLNHEIPTDILQEALPNVVAAHVMQSYIGGYGNMIQPVSACATA |
| | | AVSLEEGVDKIALGKADFVVTGAIDDIGVESVIGFGNMNATANS |
| | | EEMYGKGIDARFFSRANDRRRGGFLESQGGGTILVTRGDIAEKL |
| | | GLPVAAVVGFIHSYADGAHTSIPAPGLGALAAGLGGKDSKLVHD |
| | | LAKLGVSADDIAVVSKHDTSTNANDPNESELHNTLAHAIGRTDG |
| | | NPLFVISQKTLTGHAKGGACIFQVNGLTQLFKSGVIPANAALDCV |
| | | DPKLQRDDHMVWVRKPLRIGGGEDEFGRETAGRPVKAGLATSL |
| | | GFGHVSGFVALVHPGAFEAAVAKADGEAALEAWRERANARLAA |
| | | GQRHLEEGMMGRAALYEPIDNRRFREDHRGYDHHEVEKAMLLN |
| | | PDARLGADGYYEA |
| 3 | Wildtype | ATGACCACCTTCTTCCTCAATCGCCTAGCCACCGAGCCCCATG |
| | DNA | CGCTCGCCTTCGCCGGCCAGTCCACCCCGTGGCCGGTAGCCC |
| | | TGGCAGACCAGACCACCGATCCCAGCCTTGACGAGGCCCTGC |
| | | GCGGCCACATCGCCGCATCCAACCGACTGCTCACCCCGGTCG |
| | | CCGCCGACCTGCTGACCACCACCGGCCGTCCGGTCGACCTGT |
| | | TCGGCTTCACTCCGAACCCGGCCCGCCTCGGCGCCGCAGCCG |
| | | ACGCCACCGCGTCCGTGGAAGGCGTGGCACTGACCCAGCTC |
| | | GGCGCCCTGCTCGACCTCGAACACCTCGGCTACAGCGTGGCC |
| | | CAGGCCAAGCCGCTCGCCGTGCTCGGCCACTCCCAGGGCGT |
| | | GCTCGCCGTGCACATGGCCCGCGCCATCGAGGCCGCCGGCT |
| | | CCATCGAGGCCGCCGGCAAGACCCTTGACGAGATTCTGGCCG |
| | | TCGCCGCCCTGATCGGTGCAGCCGGCACTCGCCGCGTGCGC |
| | | GAACTGGGCCTGAACCCCAAGTATGGCGAAGCCAGTCCGATG |
| | | CTCTCCGTCAAGGGCGCCACCCGCGAACAGGTTGAGGCTCTG |
| | | GTGGCCCGCGTCAACAACGCTCGCGGCCCGATCTCCATCGCC |
| | | GTCACCAACTCCGACAACCACCACGTGCTTTCCGGCTACCCG |
| | | GAGGACCTGTCTGCCTTCGCCCTCGAAGCCGAGCGCGAACAC |
| | | CAGCATCAGGCCAAGCTGCGCGAGCAGAAGCTCCACGGCGG |
| | | CACCGTGTTCAACCCGACCCTCGAATACCTTGAGGTCACCCTG |
| | | CCCTTCCACTCGCCGCTCATGGCCGACGCCGTCGAGCAGACC |
| | | GTGGCCTGGGCCGGCGCCTGCGGCTTCGACCAGAAGCGCAC |
| | | CCGTGCGCTTGCCGAGGAAGTGCTTCTGAACCATGTGGACTG |
| | | GAACGCCCGCGTCAAGGCGCTGTTCGACGCCGCCGACCCGTC |
| | | CAAGCTGTGGATCGTGGACCTCGGCCCCGGCAACACGCTCGG |
| | | CAAGCTGATCGGCAATGTGGTGCAAGGCACCGGCATCGGCGT |
| | | GGTCGAAGCCACCACGCTGGCCGAGCGCTCCACGCTCTCCAC |
| | | GCTGGAAAGCGAGCCTGAGCGCACCCAGAACTGGAAGGCATT |
| | | CGCCCCGCGCGTCATCAACACCCCGGCCGGCGCCAAGCTGGT |
| | | CACCAAGTTCTCCAAGCTCACCGGCAAGCCGCCGGTACTGTT |
| | | GCCCCGCATGACCCCCACCACCGTGGAACCGGAAATCGTGGC |
| | | CGCCGCGGCCAACGCCGGCTACTGGGCCGAGCTCGCCGGCG |
| | | GCGGCCAGGTGACCGCCGAAGTGTTCGACCGCCACATCGCCA |
| | | CCCTCGAAGACGAGCTGGAGGAAGGCCGCACGGTCGAATTCA |
| | | ACGCGATGTTCATGGACCGTTACCTGTGGAACCTGCAGTTCG |
| | | GCTCCTCCCGCATCGTGCCCAAGAAGCGCGCATCCGGCGCGC |
| | | CGATCGACGGCGTGGTCGTCTCCGCCGGCATCCCCGAGCTG |
| | | GACGAAGCCGTGGCCCTGATCAAGTCGCTGCAGGCGGACGG |
| | | CCTGCCGTACGTGAGCTTCAAGCCCGGCACCGTGGACCAGAT |
| | | TCGTCAGGTCGTGCGCATCGCCAAGGCCGTGGCCCCCACCAC |
| | | CATCATGGTTCAGGTCGAGGGCGGTGAAGCCGGCGGCCACC |
| | | ACAGCTGGGAAGCGCTGGATGACCTGCTTGCCGCCACCTACG |
| | | CCGAAGTGCGCGCCTGTGACAACCTCGTGCTGGTCGCCGGCG |
| | | GCGGCATCGGCACTCCCGAACGCGCGGCCGACTACATTTCCG |
| | | GCCAGTGGGCCCACGCCTACGGCCTGCCCGACATGCCGGTC |
| | | GACGGCGTGCTTATCGGTACCGCCGCGATGACCGCCAAGGA |
| | | GGCTCACACCAGCCCCGAAGTCAAGCAGCTGCTCGTCAAGAC |
| | | GCCCGGTATTCCGGCCGACGCCAAGTCCGACGACGTGTTCGC |
| | | CCCGCAGGCCGCCCACTGGGTGCCGTCCGGCAAGTCCGTCG |
| | | GTGGCATGTCCTCCGGCCTGAGCCACCTGCACGCCGACATTT |
| | | ACGAGCTCGAGAACGACTCCGCCGAATGCGGCCGTCTGCTCG |
| | | TGCGCGTCATGAAGCACCCCGAAGAGCTCGACTCCCGCCGCA |
| | | AGGAAATCATCGAAGCCCTGAACAAGACCGCCAAGCCTTACTT |
| | | CGGCGACCTGGCCTCCATGACCTACCTTGAGTGGGCGCAGCG |
| | | CTTCGCCGAACTCGCCTTCCCGTGGGTCGACCCGACTTACGC |
| | | CGACCGCTTCCAGCACCTGCTGCAGCGCATCGAAGCCCGTGT |
| | | GAACGACACGGACTCCGGCGAATTCACCTCCAAGTTGTTCGC |
| | | CGCTGATGGCGTTTCTGCTGAAGAGGCAGCTGCCGCTGGCCT |
| | | GCTGACCCACGACGACATTCTCGCCGACCCGGCCCCCGCGCT |
| | | CGAAAAGCTCGCACTGGCCTACCCGCAGACCGCCGAACTCAA |
| | | GGTGGTCCCGACCGACGTGGCTTGGTTCCCGGTGCTCGTGCG |
| | | CGAATACCCCAAGCCCATGCCGTTCGTGCCCGTCATCGACAA |
| | | CGACCTGCTGCGTTGGTGGGGGCAGGACCAGCTCTGGCAGT |
| | | CCGAAGACCAGCGCTACTCCGCCGATTCCGTGCGTGCCATCC |
| | | CCGGCCCGATCTCCGTTGCCGGCATCACCACCATCGACGAGC |
| | | CGATCGCCGACATCCTCGGCCGCTTCGAAACCGCCGCCATCA |
| | | AGCGCGTGCAGGACGAACAGCAGGCCGCCGATGCCGCTGAG |
| | | AACGACGATTTCGCCGCACTTGGCGAGGCCACCAGCGCCGAA |
| | | GACTTCATCCGCAAGTCCCCGAACATCTCCTGGGTCGGCCAC |
| | | ATCACCGACAACCCGGCCTACGGCACCGCCCTGGGCGACCAG |
| | | TACTACGAGATCCGCGCGTTTGACGCCGCGGCCGGCAAGTAC |
| | | GATCTGGACATCCACCTCGACACCTACTGGGACAACGACCCG |
| | | GACGGCGGCACCTCCAAGCACGCCGTGCGCGATATCGTCATC |
| | | CCGCTCATCGTGGAAGGCGCCGAGCCCGGCCGTGTGCCGGT |
| | | CGTGGACCGCGAACGCCTGATTCCGGACGTCTACGCGATGCT |
| | | CGCCGCCACCGCAGGCATCGGCAACACCGCCATCACCGGCG |
| | | ACAAGCTCACCGAGATGCCGCAGCTTGACGGCAAGACCTCTG |
| | | ACGAGCGTCCGTTTGCTACCGCCACCGCCGCCTACACCCTCT |
| | | CCGCCAACCTCGGCTTCGACCACGAAGCGGCCACCGGCGCC |
| | | GCTCTGCCGACGACCCTGCAGCCCAGCCGCATCGCTCCCGAT |
| | | GCGCTGGTCGGCCCGGCCTGGCCGGCGATTTACACGGCGCT |
| | | CGGCTCCGTGTACGTCAAGGGCTACCCGGTGATTGAAGGCCT |
| | | GCTCAACGCCGTTCACCTCGACCACCTCATCGAACTTGAGGTG |
| | | AACGAGGAGGACCTGCTTAAGCACACCGGTGAAACCATCACC |
| | | CTGAAGTCTTGGGCTGAGGATTACTTCGAGTCCGCCTCCGGC |
| | | CGCGTGGTCACCATCCACGTGACCCACACCGCTGCCGACGGC |
| | | ACGCTGCTTGCCCGCGAAACCGAGCGTTTCGCGATTCGCGGC |
| | | CGCGTCTACTCCGATGCTTTGCCGGCAGACGCCCCCGAGTTC |
| | | GGCGACGCCGAGCACGAGGAGATCGCGCCCACGCCGCGCCG |
| | | TCTGCTGCGCCGCGTGAAGGTCGTCGCCCCACACGACATGAC |
| | | CGCCTTCGCCCGCACTTCCGGCGACTTCAACCCGATCCACAC |
| | | CTCCACTCGTGGCGCTCGCATCTCCGGTTTGGCCGCTCCGCT |
| | | TGTGCACGGCATGTGGCTGTCCGCCACCGCCCAGCACGCCGT |
| | | GCAGGCGCTGGACGAGAAGGGTGCCCACTACGAGATTGCCG |
| | | GCTGGACCTACAACATGTACGGCATGGTTCAGCTTGACGACA |
| | | CCGTCGAAATCTCCGTCGAGCGCGTCGGCAAGGTGCGCCACG |
| | | GCGGCATGACCCTCGAAGTCACCTGCCGTATCGACGGCCAGC |
| | | TCGTCTCCCGCGGCACCGCACTGGTCCGCGCGCCGCGCGCC |
| | | GCCTTCGTCTACCCCGGCCAGGGCATCCAGAAGCAGGGCATG |
| | | GTTCTGGACGAGCGTGCCAAGTCCGCCGCCGCCCGCGACGT |
| | | GTGGGAGCGCGCCGACAAGCTCACCCGCTCCAAGCTCGGCTT |
| | | CTCGATCCTGGGCCTCGTGCGCGATAACCCGAAGGAGCTCAC |
| | | CGCCAACGGCGTGACCTATCGCCACCCCGAAGGCCTGCTCAA |
| | | CCTGACGCAGTTCACGCAGGTGGCGCTCGCCACCGTGGCCTT |
| | | CGCCCAGACCGCTCGTCTGCGCGAAGCCGGCGCCGACATCTG |
| | | GCCCGCCTACTTCGCCGGCCACTCGCTCGGCGAATACAACGC |
| | | GCTGTCCGCGTTCGCCGACGTGATTCCGCTGGAAACCGTGCT |
| | | GGAGCTCGTGTTCCACCGTGGTTCCACCATGCACCACCTCATC |
| | | GAGCGTGACGCACAGGGTCGCTCCAACTACCGCATGGGTGCC |
| | | CTGCGCCCGAACCAGTTCGGTGTGGACGACGCGCACGTCAAG |
| | | GAATACGTGGAATCCGTGGCCAAGGCGTCCGGCGAATTCCTG |
| | | GAGATCGTGAACTACAACCTCGCCGGCCAGCAGTACGCCATC |
| | | GCCGGTACGATCGCCGGACTCAAGGCTCTGAAGGCCGATTCC |
| | | GCCCGCCGCGTGGCCGCATTCGGTGGCAAGCCCGCGTTCAT |
| | | GCTGGTGCCCGGCATCGATGTGCCGTTCCACTCCACACTGCT |
| | | GCGCAAGGGCGTGCCGGAATTCCGCGACAAGCTCGACGCGC |
| | | TGCTTCCCGCTTACATCGACTACCGTGGCCGTCTGGTGGACC |
| | | GCTACATCCCGAACCTGGTGGCCACCCCGTTCGAGATGACCA |
| | | AGGAATTCGCCGCCAAGATCCTCGAAGTCGTGCCGTCCGAGC |
| | | GTATCAAGGCCGTGCTCGACGACCCGGCCGTGTGGGATTCCT |
| | | ACGCCGATGACGACCAGAAGCTCGGCCGTCTGCTCTTGACCG |
| | | AACTGCTCTCCTGGCAGTTCGCCTCCCCGGTGCGTTGGATTG |
| | | AAACCCAGGCGCTGCTCTTCGGTCAGCGCGAACAGGGCGGC |
| | | CTCGGCGTGGAGGAATACGTCGAAGTCGGCCTCGGCAACGC |
| | | CCCGACCCTGGCCAACCTCGGTGCCAAGACCCTGCGTCTACC |
| | | CGAGTTCGCCGGCAACGATACGGTCGTCTACAACGTCGGCCG |
| | | CGATGAAGGCCGCGTCTACATGACCGACACCGATTCGCTGGT |
| | | TCCCGACGATGAGCCGGAAGAGGCTGCGGCCTCGGCCCCGG |
| | | TCGAAACCGCTCCTGCCGGTGGTTCCGCCGTCGCCAAGCTCG |
| | | GTTCCGGTGCCGCTCCGGCAAATGCCGTGACTGAGGCTGCTC |
| | | AGACAACGGATGGCTCCGTCGCCGGACAGTCCACTGGACTGT |
| | | CCGACTCCTCGCGCGGCCTGCAATCAGGGCAGGCCGCGGCC |
| | | GGCGCACCGTCCGGTGCCGCCGTCGCCGACCTGCCCTTCAAG |
| | | GCTTCCGATGCCATCGGCGTGCTCATGGCCTACTCCGCCAAG |
| | | GTGCGCATCGACCAGATCGGCAGCAACGACACCACTGACACC |
| | | CTGACCAACGGTGTCTCTTCTCGCCGCAACCAGCTGCTCATG |
| | | GACATCAGCTCCGAGCTGGGAGTCGCCAGCGTGGACGGTGC |
| | | TGCTGAAGCGACGCTCGACAAGCTCGCCCAAATCGTCAACAA |
| | | GGCCGCCCCGAACTACAAGCCGTTCGGTGCCGTGCTCTCCGA |
| | | GGCGCTGCGCGACCGCCTGCGCTCGCTGTTCGGTGCCGCCG |
| | | GCGTCAAGCAGCAGTACATCCGCGACCGCGTGACCAACGTAT |
| | | GGCAGCTCGGTGAAGGTTGGGTGGCCAGCGTGCTCGCCGCA |
| | | CTGCTGCTCGACACTCGCGAGGGTGCCAGCTCCCGTGGCGG |
| | | CGACCTGGCCAAGCTGCCCACCGCCGCCGTGCAGAACAAGCC |
| | | GGAAGCCGACAAGCTGATTGATGCGGCCGTTGAGGTCGTCGC |
| | | CCAGCTCAAGGGTGTTGCCGTGGCCCTGCCGTCCGCCGGTG |
| | | GTGCCGCTGGTGGCGCTGTGGTCGATTCCGCCGCGCTCGAC |
| | | GCCTTCGCCGAGAAGGTCACCGGTTCCAACGGCGTGCTCGCC |
| | | GCCACCGCCCGCTTCGTGCTGAACGAGCTCGGCGTGGCCGCT |
| | | CCGGCTCCGGAAGAATCCGAGGACGAGAACGCCGCCGTCGT |
| | | GGCCGCGGTTGAAGCCGAACTTGGCTCCGACTGGCCTAAGCA |
| | | GGTCGCTCCGCGCTTCGACGCCAACAAGGCCATCCTCTTCGA |
| | | TGATCGGTGGGCCTCCGCCCGTGAAGACCTCGCCCGCGCCTA |
| | | CTACGACAACGATCCCGCCGCCTTGAACGGCAGCTTCATCGG |
| | | CCTTGGCAAGACCATCGCCGCCGAAGCCCAGTGGTTCGCCAA |
| | | CGAGTCGGAGAGCGAAGAACTCAAGGCCGCCTTCCAGAAGG |
| | | CCGGCTCCGAGGCCCTCGAACAGGTTGCGTCCAACAAGAACG |
| | | CCTCCCGTTACGCGAATGATATCGCCATCGTCACCGGCGTGA |
| | | GCCCGAACTCGATTGCCGCTCAGGTGGTCGAAGGCCTGCTCG |
| | | CAGGCGGTGCCACTGTGGTGGCCACCTCGCACAGCTTCAAGC |
| | | CGTCCATTAAGGCTTGGGCTAAGCAGGCCTACCGCGAGCACG |
| | | CTACCGGCAACGCCAAGCTGTGGCTGGTTCCGGCCAACCTCT |
| | | CCAGCTACCGCGACGTCGACGCACTGGTCGACTGGGTGGGC |
| | | CACGAGCAGAAGAAGACCTCCGGTGCTACCACCACGATTCTG |
| | | AAGCCCGCGTGGGAGCCGACTCTGTTCTTCCCGTTCGCCGCT |
| | | CCGCCGGTCCATGGCACGCTCGCCGACTCCGGCGACCTGTTC |
| | | GAATCGCAGGCCCGACTGATGCTGTGGGGCGTGGAACGCGC |
| | | CATCGCCGGCTTCAGCCACATCGGTGCCGACACCAACGTGCA |
| | | GCACAAGCTCCACGTGGTGCTGCCCGGTTCCCCGAACCGCGG |
| | | CGTATTCGGCGGCGACGGTGCCTACGGCGAAGTCAAGAGCG |
| | | CCTTTGACGCCATCGTCAACCGTGCCCGCGCTGAAAAGGTGT |
| | | GGTCGAGCCGCGTCACCTTCGCTCACCCGAAGATCGGCTGGG |
| | | TGCGTGGCACCGGCCTGATGGTCGGTAACGACCCGCTGGTCG |
| | | CCGTCGTTGAGCGTCACGGCATCCGCACCTACTCCACCGCAC |
| | | AGATTGCGGCCAAGCTGCTCGACCTGTGCACTGCCGAATCGC |
| | | GCGAGCAGGCCCTCAAGGCCCCGCTCGATGTGGACCTGACC |
| | | GGTGGCCTCGGCTCCGAGCCCATCGACATCAAGGCCCTGCGT |
| | | GCGGAGGCCATGGCCGACGCCGAGAAGGAAGCTGCTGCGGC |
| | | CTCGTCTCAGGAAACGGATGGCTCCGTCGCCGGAAAGTCCAC |
| | | TGGACTGTCCGACTCCTCGCGTGGGCAGCAAATCAAGGCGCT |
| | | GCCCACACCGATCGTCACCAAGCAGGCTCCGGTCGACTTGAA |
| | | CGATTGGACCAACGTCACTGCCAAGCCGGAAGACGAAATCGT |
| | | CATCGTTTCCGTCGGTGAGCTGGGCCCGTGGGGCTCCGGTCG |
| | | TACGCGCGCCCAGGCCGAGCTCGGCATCCACTCGGACGGCA |
| | | CCGTCGACCTCAGCGCCGGCGCAGTGCTCGAACTCGCGTGGA |
| | | ACATGGGCCTGCTGACTTGGGCCGACAGCCCGAAGCCCGGCT |
| | | GGTACGACACCGACGGCAACCTCGTGCCCGAAGAGGACATCG |
| | | CCGAGCGCTACCACGACGAGGTCGTGGCCCGCTCCGGCATCC |
| | | GCCCGTTCGAGGAAGGCATGGGCAACGACTACAAGGACGGT |
| | | GCTGACGAAGAGGAAGCCGAGGTCTTCCTCGACCACGACGTC |
| | | ACCTTCTCTGTGCCCACCCGCGAAGTCGCCGCCGAATACGTC |
| | | AAGTTGGACGAGGCTCACACCACCATCGCCCCGGATGAGGAA |
| | | TCCGGCGAATGGAACGTGACCCGCCACGCCGGCTCGATGATC |
| | | CGCGTGCCGCGCCGCGCCACCATGACCCGCACAGTCGGCGG |
| | | CCAGTTCCCGAAGGGCTTCGACCCGACCCGATGGGGCATTCC |
| | | CGCCTCGATGGTCGGCGACGTCGACAAGATCGCCCTGTGGAA |
| | | CATCGTCACCACTGTGGACGCCTACCTGGGCGCCGGCTTCAC |
| | | CCCGGCCGAAATCCTCGAGTCCATCCACCCCTCGCTGGTGGC |
| | | TTCGACCCAGGGCACCGGCTTCGGTGGCATGATGTCGATGCG |
| | | CAAGCTGTACCTCGACCGCTTCCTCAACCACGAGATCCCGACC |
| | | GACATCCTGCAGGAAGCCCTGCCGAACGTGGTCGCGGCTCAC |
| | | GTGATGCAGTCCTACATCGGCGGCTACGGCAACATGATTCAG |
| | | CCGGTCTCCGCCTGCGCCACCGCAGCCGTCTCCCTTGAAGAG |
| | | GGCGTCGACAAGATCGCCCTCGGCAAGGCCGACTTCGTGGTC |
| | | ACCGGTGCGATCGACGACATCGGCGTGGAATCCGTGATCGGT |
| | | TTCGGCAACATGAACGCCACCGCCAACTCGGAGGAAATGTAT |
| | | GGCAAGGGTATCGACGCGCGCTTCTTCAGCCGCGCGAACGAT |
| | | CGTCGCCGTGGCGGCTTCCTGGAATCCCAGGGCGGCGGCAC |
| | | GATCCTCGTGACCCGCGGTGACATCGCCGAAAAGCTCGGCCT |
| | | GCCGGTTGCGGCCGTGGTCGGCTTCATCCACTCCTACGCGGA |
| | | TGGCGCTCACACCTCGATCCCGGCTCCGGGCCTCGGTGCCCT |
| | | GGCTGCCGGCCTCGGCGGCAAGGACTCCAAGCTCGTGCACG |
| | | ACCTGGCTAAGCTCGGCGTCTCAGCCGACGACATCGCCGTGG |
| | | TCTCCAAGCACGACACGTCCACCAACGCCAACGACCCGAACG |
| | | AATCCGAGCTGCACAACACGCTGGCCCACGCCATCGGCCGCA |
| | | CCGACGGCAACCCGCTGTTCGTGATCTCGCAGAAGACGCTTA |
| | | CCGGCCACGCCAAGGGTGGTGCCTGCATCTTCCAGGTCAACG |
| | | GTCTGACGCAGCTGTTCAAGTCCGGCGTGATTCCGGCGAACG |
| | | CGGCGCTCGACTGCGTGGATCCGAAGCTCCAGCGCGACGAC |
| | | CACATGGTCTGGGTTAGGAAGCCCCTGCGCATCGGCGGTGGT |
| | | GAGGACGAGTTCGGTCGCGAGACCGCTGGTCGCCCGGTCAA |
| | | GGCCGGTCTGGCCACCTCGCTCGGCTTCGGACATGTTTCCGG |
| | | CTTCGTGGCTCTGGTCCATCCGGGTGCCTTCGAGGCGGCCGT |
| | | GGCCAAGGCCGATGGTGAGGCCGCACTGGAAGCTTGGCGTG |
| | | AGCGTGCCAACGCCCGCCTGGCTGCCGGCCAGCGTCACCTTG |
| | | AGGAAGGCATGATGGGTCGCGCCGCGCTCTACGAGCCGATC |
| | | GACAACCGTCGCTTCCGCGAGGATCACCGTGGCTACGACCAC |
| | | CACGAGGTCGAAAAGGCGATGCTCCTCAACCCGGACGCCCGC |
| | | CTCGGTGCCGACGGCTACTACGAGGCCTGA |
| 4 | Wildtype | MTTFFLNRLATEPHALAFAGQSTPWPVALADQTTDPSLDEALRG |
| | protein | HIAASNRLLTPVAADLLTTTGRPVDLFGFTPNPARLGAAADATAS |
| | | VEGVALTQLGALLDLEHLGYSVAQAKPLAVLGHSQGVLAVHMA |
| | | RAIEAAGSIEAAGKTLDEILAVAALIGAAGTRRVRELGLNPKYGE |
| | | ASPMLSVKGATREQVEALVARVNNARGPISIAVTNSDNHHVLSG |
| | | YPEDLSAFALEAEREHQHQAKLREQKLHGGTVFNPTLEYLEVTLP |
| | | FHSPLMADAVEQTVAWAGACGFDQKRTRALAEEVLLNHVDWN |
| | | ARVKALFDAADPSKLWIVDLGPGNTLGKLIGNVVQGTGIGVVEA |
| | | TTLAERSTLSTLESEPERTQNWKAFAPRVINTPAGAKLVTKFSKL |
| | | TGKPPVLLPRMTPTTVEPEIVAAAANAGYWAELAGGGQVTAEVF |
| | | DRHIATLEDELEEGRTVEFNAMFMDRYLWNLQFGSSRIVPKKRA |
| | | SGAPIDGVVVSAGIPELDEAVALIKSLQADGLPYVSFKPGTVDQI |
| | | RQVVRIAKAVAPTTIMVQVEGGEAGGHHSWEALDDLLAATYAE |
| | | VRACDNLVLVAGGGIGTPERAADYISGQWAHAYGLPDMPVDGV |
| | | LIGTAAMTAKEAHTSPEVKQLLVKTPGIPADAKSDDVFAPQAAH |
| | | WVPSGKSVGGMSSGLSHLHADIYELENDSAECGRLLVRVMKHP |
| | | EELDSRRKEIIEALNKTAKPYFGDLASMTYLEWAQRFAELAFPWV |
| | | DPTYADRFQHLLQRIEARVNDTDSGEFTSKLFAADGVSAEEAAA |
| | | AGLLTHDDILADPAPALEKLALAYPQTAELKVVPTDVAWFPVLVR |
| | | EYPKPMPFVPVIDNDLLRWWGQDQLWQSEDQRYSADSVRAIP |
| | | GPISVAGITTIDEPIADILGRFETAAIKRVQDEQQAADAAENDDF |
| | | AALGEATSAEDFIRKSPNISWVGHITDNPAYGTALGDQYYEIRAF |
| | | DAAAGKYDLDIHLDTYWDNDPDGGTSKHAVRDIVIPLIVEGAEP |
| | | GRVPVVDRERLIPDVYAMLAATAGIGNTAITGDKLTEMPQLDGK |
| | | TSDERPFATATAAYTLSANLGFDHEAATGAALPTTLQPSRIAPDA |
| | | LVGPAWPAIYTALGSVYVKGYPVIEGLLNAVHLDHLIELEVNEED |
| | | LLKHTGETITLKSWAEDYFESASGRVVTIHVTHTAADGTLLARET |
| | | ERFAIRGRVYSDALPADAPEFGDAEHEEIAPTPRRLLRRVKVVAP |
| | | HDMTAFARTSGDFNPIHTSTRGARISGLAAPLVHGMWLSATAQ |
| | | HAVQALDEKGAHYEIAGWTYNMYGMVQLDDTVEISVERVGKVR |
| | | HGGMTLEVTCRIDGQLVSRGTALVRAPRAAFVYPGQGIQKQGM |
| | | VLDERAKSAAARDVWERADKLTRSKLGFSILGLVRDNPKELTAN |
| | | GVTYRHPEGLLNLTQFTQVALATVAFAQTARLREAGADIWPAYF |
| | | AGHSLGEYNALSAFADVIPLETVLELVFHRGSTMHHLIERDAQG |
| | | RSNYRMGALRPNQFGVDDAHVKEYVESVAKASGEFLEIVNYNLA |
| | | GQQYAIAGTIAGLKALKADSARRVAAFGGKPAFMLVPGIDVPFH |
| | | STLLRKGVPEFRDKLDALLPAYIDYRGRLVDRYIPNLVATPFEMTK |
| | | EFAAKILEVVPSERIKAVLDDPAVWDSYADDDQKLGRLLLTELLS |
| | | WQFASPVRWIETQALLFGQREQGGLGVEEYVEVGLGNAPTLAN |
| | | LGAKTLRLPEFAGNDTVVYNVGRDEGRVYMTDTDSLVPDDEPEE |
| | | AAASAPVETAPAGGSAVAKLGSGAAPANAVTEAAQTTDGSVAG |
| | | QSTGLSDSSRGLQSGQAAAGAPSGAAVADLPFKASDAIGVLMA |
| | | YSAKVRIDQIGSNDTTDTLTNGVSSRRNQLLMDISSELGVASVD |
| | | GAAEATLDKLAQIVNKAAPNYKPFGAVLSEALRDRLRSLFGAAG |
| | | VKQQYIRDRVTNVWQLGEGWVASVLAALLLDTREGASSRGGDL |
| | | AKLPTAAVQNKPEADKLIDAAVEVVAQLKGVAVALPSAGGAAGG |
| | | AVVDSAALDAFAEKVTGSNGVLAATARFVLNELGVAAPAPEESE |
| | | DENAAVVAAVEAELGSDWPKQVAPRFDANKAILFDDRWASARE |
| | | DLARAYYDNDPAALNGSFIGLGKTIAAEAQWFANESESEELKAA |
| | | FQKAGSEALEQVASNKNASRYANDIAIVTGVSPNSIAAQVVEGL |
| | | LAGGATVVATSHSFKPSIKAWAKQAYREHATGNAKLWLVPANL |
| | | SSYRDVDALVDWVGHEQKKTSGATTTILKPAWEPTLFFPFAAPP |
| | | VHGTLADSGDLFESQARLMLWGVERAIAGFSHIGADTNVQHKL |
| | | HVVLPGSPNRGVFGGDGAYGEVKSAFDAIVNRARAEKVWSSRV |
| | | TFAHPKIGWVRGTGLMVGNDPLVAVVERHGIRTYSTAQIAAKLL |
| | | DLCTAESREQALKAPLDVDLTGGLGSEPIDIKALRAEAMADAEK |
| | | EAAAASSQETDGSVAGKSTGLSDSSRGQQIKALPTPIVTKQAPV |
| | | DLNDWTNVTAKPEDEIVIVSVGELGPWGSGRTRAQAELGIHSD |
| | | GTVDLSAGAVLELAWNMGLLTWADSPKPGWYDTDGNLVPEEDI |
| | | AERYHDEVVARSGIRPFEEGMGNDYKDGADEEEAEVFLDHDVT |
| | | FSVPTREVAAEYVKLDEAHTTIAPDEESGEWNVTRHAGSMIRVP |
| | | RRATMTRTVGGQFPKGFDPTRWGIPASMVGDVDKIALWNIVTT |
| | | VDAYLGAGFTPAEILESIHPSLVASTQGTGFGGMMSMRKLYLDR |
| | | FLNHEIPTDILQEALPNVVAAHVMQSYIGGYGNMIQPVSACATA |
| | | AVSLEEGVDKIALGKADFVVTGAIDDIGVESVIGFGNMNATANS |
| | | EEMYGKGIDARFFSRANDRRRGGFLESQGGGTILVTRGDIAEKL |
| | | GLPVAAVVGFIHSYADGAHTSIPAPGLGALAAGLGGKDSKLVHD |
| | | LAKLGVSADDIAVVSKHDTSTNANDPNESELHNTLAHAIGRTDG |
| | | NPLFVISQKTLTGHAKGGACIFQVNGLTQLFKSGVIPANAALDCV |
| | | DPKLQRDDHMVWVRKPLRIGGGEDEFGRETAGRPVKAGLATSL |
| | | GFGHVSGFVALVHPGAFEAAVAKADGEAALEAWRERANARLAA |
| | | GQRHLEEGMMGRAALYEPIDNRRFREDHRGYDHHEVEKAMLLN |
| | | PDARLGADGYYEA |

In the context of the present invention, a mutation in the gene (gene mutation) is to be understood as an alteration in the nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift.

In the present description and claims the conventional one-letter code for nucleotides is used following the analogous principles as described for amino acids nomenclature supra.

Algorithms for aligning sequences and determining the degree of sequence identity between them are well known in the art. For the purpose of the present invention a process may be carried out for aligning nucleotide sequences using blastn as provided by the National Center for Biotechnology Information (NCBI) on https://blast.ncbi.nlm.nih.gov applying standard parameter.

### EXAMPLES

### Example 1—Medium Composition Affects the Stability of Bifidobacteria

### Bacterial Strains

The strains B. animalis subsp. lactis BB-12® (herein also called BA, DSM 15954) and B. longum subsp. longum BB-46 (herein also called BL, DSM 15955) were obtained from the Chr. Hansen Culture Collection.

### Strain Maintenance

Twenty percent (v/v) glycerol stocks of the strains were stored at −80 °C. The glycerol stocks were prepared from anaerobic batch cultivations in chemically defined medium (CDM) with 10 g L⁻¹ sucrose as carbon source.

### Batch Fermentations

The strains were cultivated in triplicates in 200 mL medium with 10 g L⁻¹ sucrose in anaerobic pH-controlled (pH 6.5) batch fermentations in a DASGIP Parallel Bioreactor System (Eppendorf, Hamburg, Germany) at 37 °C. Two different media compositions were tested in this study, with unaltered CDM for bifidobacteria used as reference medium. The CDM-Ref contains 1 mL L⁻¹ Tween® 80 (CAS nr.: 9005-65-6). Besides in the original CDM (CDM-Ref), cultivations were conducted in CDM without Tween® 80 (CDM-no-Tween).

### Assessment of Viability During Short-Term Storage

The robustness and stability of BA and BL were assessed in short-term storage tests under suboptimal conditions. From each replicate, samples of 5 mL cell culture were harvested in the stationary growth phase at 11000 rpm and 5 °C for 10 min. To avoid a loss of cells only 4 mL of cell supernatant was discarded. The cell pellet was resuspended after adding 4 mL of peptone saline solution (8.5 g L⁻¹ NaCl, 1 g L⁻¹ peptone, pH 7). The survival of cells was assessed after storing them protected from light for 28 days at 2-4 °C. After 14 days the cells were vortexed for 1 min at 24000 rpm to avoid cell aggregation. Before and after storage for 28 days, the number of viable cells was determined by colony forming units (CFU) applying the pour plate method. The loss of viable cells was calculated as log 10 (CFU_{before storage}/CFU_{after storage}) and was used as a measure for the survivability of the strains.

### Fatty Acid Analysis

The relative fatty acid composition of BA and BL in the stationary phase was assessed using gas chromatography mass spectrometry (GC-MS) system (ISQ-LT, Thermo Scientific, USA) equipped with a ZB-FAME column (20 m×0.18 mm×0.15 μm, Phenomenex, Torrance, CA, USA). As a standard mix, the bacterial acid methyl ester (BAME) mix (47080-U, Sigma Aldrich) was used. To test the significance of the difference between means of fatty acids as compared to the profile observed in CDM-Ref, multiple t-testing was applied (Holm-Šídák method, unpaired, Welch correction). The statistical analysis was run using GraphPad Prism version 9.3.1, GraphPad Software, San Diego, CA, USA.

### Growth Profiling

The optical density of the culture broth at 600 nm (OD600) was determined using an Ultrospec 10 spectrophotometer (GE Healthcare, USA).

### Genome Sequencing and SNP Analysis

Genome sequencing was performed as described in: Schöpping M, Gaspar P, Neves A R, et al (2021) Identifying the essential nutritional requirements of the probiotic bacteria Bifidobacterium animalis and Bifidobacterium longum through genome-scale modeling. npj Syst Biol Appl 7:1-15. Single nucleotide polymorphisms (SNPs) analysis was performed in CLC Genomics Workbench (22.0.1), setting the minimum frequency at 85%.

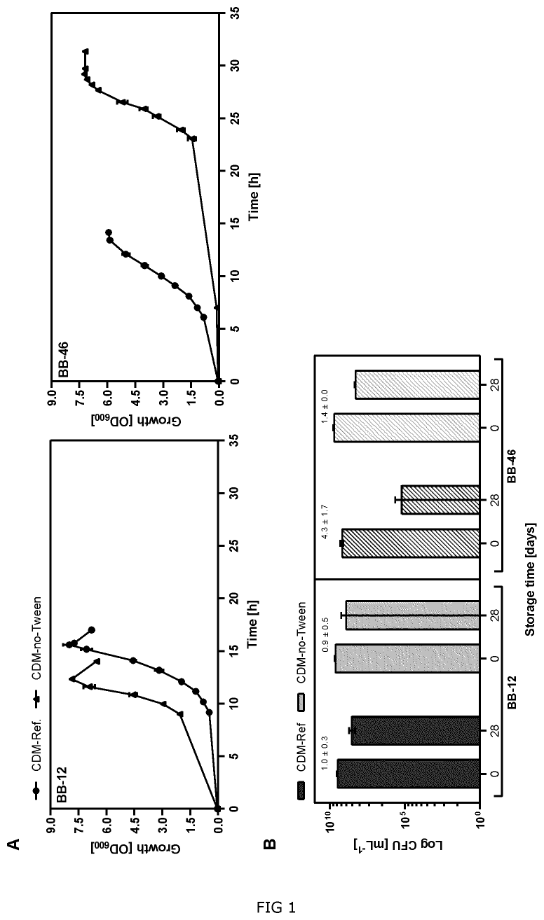

Growth characteristics. The metabolism and physiology of BA and BL were characterized in CDM with and without an exogenous fatty acid source. Growth and survivability of the two strains were found to be affected by the formulation of the medium (FIG. 1). Cultivations were conducted under anaerobic conditions (20% CO₂ and 80% N₂) at 37 °C and pH 6.5. CDM-Ref: reference chemically defined medium with 1 mL L⁻¹ Tween® 80, CDM-no-Tween: CDM without Tween® 80.

BL showed a very long lag phase of around 20 h when cultivated in CDM-no-Tween (FIG. 1A). This suggested that BL used exogenous fatty acids in the preculture and was challenged by the shift to de novo fatty acid synthesis.

Sequencing of BL grown in CDM-no-Tween. Due to the long lag phase of BL in CDM-no-Tween, cells cultivated under that condition were genome sequenced. Genome sequencing revealed a single nucleotide polymorphism (SNP) in its microbial type I fatty acid synthase (FAS) gene (13242_09035). At position 1219 of the nucleotide sequence of the gene, a cytosine was exchanged by an adenine. FAS is a multifunctional enzyme responsible for de novo fatty acid synthesis [Schweizer E, Hofmann J (2004) Microbial type I fatty acid synthases (FAS): major players in a network of cellular FAS systems. Microbiol Mol Biol Rev 68:501-17, table of contents.]. The mutation in the gene resulted in the substitution of arginine for serine at position 407 of the protein sequence. Multiple sequence analysis revealed that BA's FAS gene (BIF_00783) possesses a glycine at the corresponding position, which is as serine a small amino acid. The surrounding amino acids in the FAS genes of BA and BL were found to be highly similar.

To assess if the described mutation is recurrent, the cultivation of BL in CDM-no-Tween was repeated in triplicates and cells from all replicates were genome sequenced. In all replicates, the cells showed the same SNP in the FAS gene as described above. Moreover, the mutation was shown to be genetically stable when the derivates were subsequently cultivated in CDM-Ref which contained Tween® 80. In the following text the derivate of BL will be referred to as FAS-mutant of BL.

Survivability. The formulation of the growth medium was found to influence the robustness and stability of BA and BL, which were assessed in short-term storage tests (FIG. 1B). The viability of the strains was assessed applying colony forming units (CFU) counts before and after storage (aerobic conditions, pH=7, 2-4 °C, 28 days). 0: CFU count before storage, 28: CFU count after storage for 28 days. The values above the bars correspond to the log loss of viable cells during storage measured in CFU.

In both media used for their propagation, BA demonstrated greater survival than BL (FIG. 1B). BA exhibited the similar survivability when produced in CDM-Ref and CDM-no-Tween (FIG. 1B). In contrast, the FAS-mutant of BL grown in CDM-no-Tween showed better survival than BL produced in CDM-Ref (FIG. 1B).

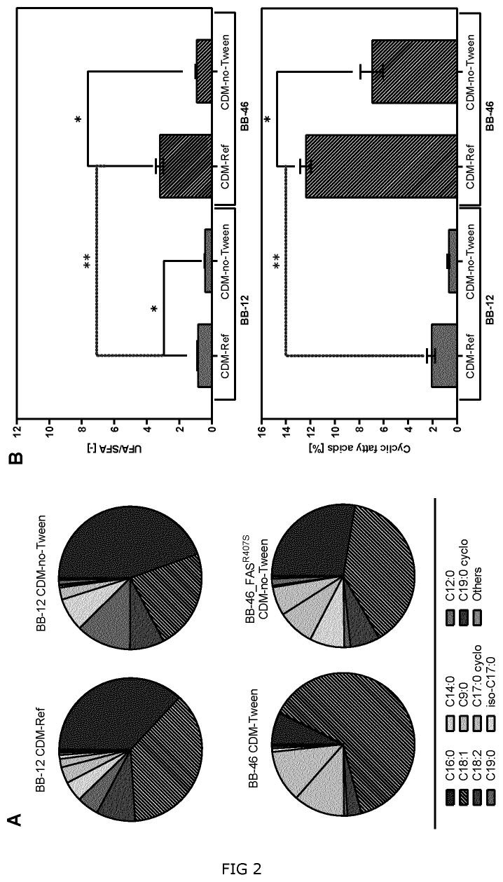

Fatty acid profile. Another cellular characteristic that was compared across the two media, was the cell membrane fatty acid composition of BA, BL and the FAS mutant of BL, respectively. The fatty acid composition of the strains was assessed once entered the stationary phase. For the comparison, the fatty acid profile of the strains in CDM-Ref was used as reference. In CDM-no-Tween, significant changes in the fatty acid composition were observed for BA and for the FAS-mutant of BL (FIG. 2). FIG. 2A shows the content of individual fatty acids in the cell membrane of BB-12® and BB-46. Each value gives the mean of a triplicate. FIG. 2B shows fatty acid profile characteristics of BB-12® and BB-46. Each value gives the mean of a triplicate±standard deviation. UFA/SFA: ratio of unsaturated to saturated fatty acids. The UFA/SFA was calculated without considering cyclic fatty acids. The significance of the difference of means were tested applying multiple unpaired t-test with CDM-Ref serving as reference. One asterisk: p_{adj}<0.05: Two asterisks: p_{adj}<0.005: **. In the absence of an exogenous fatty acid source, thus in CDM-no-Tween, a significantly lower oleic acid (C18:1) content and a significantly higher palmitic acid (C16:0) content was detected in the cell membrane of BA and in that of the FAS-mutant of BL (FIG. 2A). The relative increase of the palmitic acid content in the cell membrane of BA was only about 8%, while it was over 20% in the cell membrane of the FAS-mutant of BL. Moreover, the myristic acid (C14:0) content was significantly higher in both strains in CDM-no-Tween (FIG. 2). The FAS-mutant of BL exhibited further a higher percentage of linoleic acid (C18:2) but a lower percentage of cis-9,10-methylenehexadecanoate (C17:0 cyclo) as compared to BL in CDM-Ref (FIG. 2A). Additional significant differences in the fatty acid profile of BA between CDM-no-Tween and CDM-Ref included a higher nonadecanoic acid (C19:0) and a lower 15-metyhlhexadecanoate (iso-C17:0) content (FIG. 2A).

Despite significant differences in BA's cell membrane composition in CDM-no-Tween as compared to CDM-Ref, the fatty acid profile of the strain was consistently dominated by oleic acid and palmitic acid. This is in line with the prevalence of the two fatty acids in the cell membrane of other Bifidobacterium strains [Ruiz L, Sánchez B, Ruas-Madiedo P, et al (2007) Cell envelope changes in Bifidobacterium animalis subsp. lactis as a response to bile. FEMS Microbiol Lett 274:316-322., and Louesdon S, Charlot-Rougé S, Tourdot-Maréchal R, et al (2015) Membrane fatty acid composition and fluidity are involved in the resistance to freezing of Lactobacillus buchneri and Bifidobacterium longum. Microb Biotechnol 8:311-318.]. In contrast, the fatty acid profile of BL in CDM-Ref was by far dominated by oleic acid (>60%) and the content of palmitic acid was below 7.5% (FIG. 2A). Since the primary fatty acid in Tween® 80 is oleic acid (Product Information Sheet, Sigma, CAS nr.: 9005-65-6), the dominance of oleic acid in the cell membrane of BL at these conditions suggests a strong utilization of exogenous fatty acids.

In addition, significant differences between the fatty acid profile of BA and BL were observed in CDM-Ref. The comparison revealed a significant difference in the percentage of seven fatty acids in the cell membrane of both strains. In contrast, the fatty acid profile of the FAS-mutant of BL in CDM-no-Tween was more similar to that of BA in CDM-Ref, showing only a significantly lower C16:0 content. However, this difference (25%) was considerably smaller than when BL was cultivated in the other growth media (80-96%) (FIG. 2A).

Overall, the observed changes in the fatty acid profile of both strains in CDM-no-Tween as compared to CDM-Ref confirmed that the cell membrane composition of both strains is affected by the supply of exogenous fatty acids in the media. The smaller changes observed in the fatty acid profile of BA when propagated in CDM-no-Tween suggest that that the strain synthesizes a high fraction of cell membrane fatty acids de novo regardless of the presence of fatty acids in the medium. In contrast, the long lag phase, and the natural selection of a FAS mutant in the medium without Tween® 80, proposed that BL cannot grow in the absence of an exogenous fatty acid source. Instead, the mutation in the FAS gene is most likely required for the ability of the BL derivate to grow in CDM without Tween® 80. The bifidobacterial FAS has not been characterized yet, however, protein sequence analysis revealed that the mutation in the BL derivate lies in an enoyl reductase domain of the yeast-type FAS subunit beta (COG4981). The functional consequences of the mutation remain to be investigated.

Correlating the fatty acid profiles with the survivability of the two strains across growth conditions, suggests that an almost equal content of palmitic acid and oleic acid, accompanied with a UFA/SFA of around 1, contribute to an enhanced robustness and stability in both Bifidobacterium strains (FIG. 1 and FIG. 2). A high content of SFA in the cell membrane of bacteria has been suggested to contribute to a reduced membrane fluidity, which may promote the strains' tolerance to stressors [Louesdon S, Charlot-Rougé S, Tourdot-Maréchal R, et al (2015) Membrane fatty acid composition and fluidity are involved in the resistance to freezing of Lactobacillus buchneri and Bifidobacterium longum. Microb Biotechnol 8:311-318., Beales N (2004) Adaptation of microorganisms to cold temperatures, weak acid preservatives, low pH, and osmotic stress: A review. Compr Rev Food Sci Food Saf 3:1-20.]. The described characteristics are fulfilled by BA's cell membrane in both growth media, whereas they were not observed for BL but for the FAS-mutant of BL in CDM-no-Tween.

Taken together, our results highlight that exogenous fatty acids can have an undesirable effect on the fatty acid profile of Bifidobacterium strains, potentially reducing the strains' robustness and stability. To avoid variations in the fatty acid supply in complex production medium due to batch-to batch variations of undefined medium components, the use of a CDM in industrial-scale production might be advantageous.

In line with our results that clearly suggest a key role of the cell membrane composition in bifidobacterial stress tolerance, the fatty acid profile of Bifidobacterium strains has previously been found to change along with the acquisition of stress tolerance of strains in adaptive laboratory evolution experiments and in response to stress exposure [Ruiz L, Sánchez B, Ruas-Madiedo P, et al (2007) Cell envelope changes in Bifidobacterium animalis ssp. lactis as a response to bile. FEMS Microbiol Lett 274:316-322., Louesdon S, Charlot-Rougé S, Tourdot-Maréchal R, et al (2015) Membrane fatty acid composition and fluidity are involved in the resistance to freezing of Lactobacillus buchneri and Bifidobacterium longum. Microb Biotechnol 8:311-318, Yang X, Hang X, Zhang M, et al (2015) Relationship between acid tolerance and cell membrane in Bifidobacterium, revealed by comparative analysis of acid-resistant derivatives and their parental strains grown in medium with and without Tween 80. Appl Microbiol Biotechnol 99:5227-5236., Wei Y, Gao J, Liu D, et al (2019) Adaptational changes in physiological and transcriptional responses of Bifidobacterium longum involved in acid stress resistance after successive batch cultures. Microb Cell Fact 18:156.] For example, the bile resistant derivate B. animalis subsp. lactis IPLA 4549 showed a different fatty acid profile as compared to its parental strain, with a lower UFA/SFA in the absence and a higher UFA/SFA in the presence of bile [Ruiz L, Sánchez B, Ruas-Madiedo P, et al (2007) Cell envelope changes in Bifidobacterium animalis subsp. lactis as a response to bile. FEMS Microbiol Lett 274:316-322]. Contrary to our findings, the acid tolerance of the acid-resistant derivates B. longum JDY1017dpH and B. breve BB8dpH was especially enhanced when the strains were grown in the presence of Tween® 80 and showed an increased oleic acid content and UFA/SFA, compared to their parental strains. Nevertheless, the acid-resistant strains exhibited a reduced membrane fluidity, most likely due to the presence of cyclic fatty acids and a higher mean fatty acid chain length in the derivates' cell membrane [Yang X, Hang X, Zhang M, et al (2015) Relationship between acid tolerance and cell membrane in Bifidobacterium, revealed by comparative analysis of acid-resistant derivatives and their parental strains grown in medium with and without Tween 80. Appl Microbiol Biotechnol 99:5227-5236.]. Moreover, in B. longum R0175 increased survival of stationary phase cells than exponential phase cells during freezing was suggested to be associated with decreased membrane fluidity, due to a relative increase of saturated fatty acids in the strain's membrane [Louesdon S, Charlot-Rougé S, Tourdot-Marechal R, et al (2015) Membrane fatty acid composition and fluidity are involved in the resistance to freezing of Lactobacillus buchneri and Bifidobacterium longum. Microb Biotechnol 8:311-318.].

In agreement with our results, a higher palmitic acid and a lower oleic acid content was detected in the cell membrane of the hydrogen peroxide (H₂O₂)-resistant B. animalis subsp. lactis strain BL-04 as compared to a genetically closely related, publicly available, H₂O₂-sensitive B. animalis subsp. lactis strain when grown in medium with Tween® 80 [Oberg T S, Steele J L, Ingham S C, et al (2011) Intrinsic and inducible resistance to hydrogen peroxide in Bifidobacterium species. J Ind Microbiol Biotechnol 38:1947-1953., and Oberg T S, Ward R E, Steele J L, Broadbent R (2013) Genetic and physiological responses of Bifidobacterium animalis subsp. lactis to hydrogen peroxide stress. J Bacteriol 195:3743-3751.]. The stress resistance of this H₂O₂-sensitive strain increased when the strain was grown in the absence of any fatty acid source and de novo fatty acid synthesis resulted in a higher palmitic acid and lower oleic acid content in the cell membrane of the strain. The authors suspected the higher cyclic fatty acid content in the cell membrane of BL-04 to be decisive for the strain's higher H₂O₂ tolerance as compared to the H₂O₂-sensitive strain in the presence of Tween® 80. However, this conclusion is in contradiction to our results, showing a higher cyclic acid content in the cell membrane of the stress-sensitive strain BL than in BA's cell membrane (FIG. 2B). In addition, a 54 bp deletion (incorrectly stated as 45 bp deletion in reference) in a gene encoding a long-chain fatty acid CoA ligase in the genome of BL-04 was suggested to limit the H₂O₂-sensitive strain's ability to use exogenous fatty acids in comparison to the strain. However, this conclusion can be challenged. First, like BA and BL, BL-04 and the H₂O₂-sensitive strain harbor four long-chain fatty acid CoA ligase genes that may facilitate the use of exogenous fatty acids. Moreover, BA, which in our study showed less pronounced changes in its fatty acid profile across growth media than BL, possesses exactly the same set of long-chain fatty acid CoA ligase genes as the H₂O₂-sensitive strain but not the gene with the 54 bp deletion as detected in BL-04. Both findings disagree with the suggested hypothesis.

1. An isolated strain of Bifidobacterium longum characterized in that the arginine in the wild type fatty acid synthase (FAS) gene is substituted to an amino acid selected from the group consisting of glycine and serine.

2. An isolated strain of Bifidobacterium longum characterized in that
a. the FAS gene sequence comprises SEQ ID NO:1; and
b. it has ability to grow in the absence of an exogenous fatty acid source.

3. An isolated strain of Bifidobacterium longum characterized in that
a. the FAS protein sequence comprises SEQ ID NO:2; and
b. it has ability to grow in the absence of an exogenous fatty acid source.

4. An isolated strain according to claim 1 which has a low ratio of unsaturated to saturated fatty acids compared to the wild type strain, such as 1:1.

5. An isolated strain according to claim 1 which is selected from the group consisting of B. longum subsp. longum BB-46, B. longum subsp. longum AF13-34 or B. longum subsp. longum AF13-41.

6. An isolated strain according to claim 5 which is the B. longum subsp. longum BB-46, deposited as DSM 15955.

7. A probiotic product comprising an isolated strain according to claim 1 and a cryoprotectant.

8. A probiotic product according to claim 7 wherein the cryoprotectant is a saccharide.

9. (canceled)

10. A method of manufacturing a Bifidobacterium longum strain according to claim 1, wherein the growth medium is a medium without an exogenous fatty acid source, such as Tween® 80.

11. A method for the prevention, alleviation of symptoms, and/or treatment of an intestinal inflammatory condition such as IBD and IBS, a liver disease such as NAFLD, NASH, cirrhosis, or alcohol-related liver disease, a metabolic disorder such as metabolic syndrome, insulin resistance, type 2 diabetes, obesity, cardiovascular atherosclerosis, an autoimmune disease, such as celiac disease, type 1 diabetes, multiple sclerosis or rheumatoid arthritis, and/or a mental condition such as major depressive disorder, a mood disorder, a cognitive disorder, chronic fatigue syndrome, or anxiety, the method comprising administering a therapeutically effective dose of an isolated strain according to claim 1 to an individual in need thereof.


SEQUENCE LISTING (4 sequences)

SEQ ID NO: 1 - DNA, 9519 bp, synthetic construct
  source 1..9519: mol_type = other DNA
atgaccaccttcttcctcaatcgcctagccaccgagccccatgcgctcgccttcgccggccagtccaccccgtggccggtagccctggcagaccagaccaccgatcccagccttgacgaggccctgcgcggccacatcgccgcatccaaccgactgctcaccccggtcgccgccgacctgctgaccaccaccggccgtccggtcgacctgttcggcttcactccgaacccggcccgcctcggcgccgcagccgacgccaccgcgtccgtggaaggcgtggcactgacccagctcggcgccctgctcgacctcgaacacctcggctacagcgtggcccaggccaagccgctcgccgtgctcggccactcccagggcgtgctcgccgtgcacatggcccgcgccatcgaggccgccggctccatcgaggccgccggcaagacccttgacgagattctggccgtcgccgccctgatcggtgcagccggcactcgccgcgtgcgcgaactgggcctgaaccccaagtatggcgaagccagtccgatgctctccgtcaagggcgccacccgcgaacaggttgaggctctggtggcccgcgtcaacaacgctcgcggcccgatctccatcgccgtcaccaactccgacaaccaccacgtgctttccggctacccggaggacctgtctgccttcgccctcgaagccgagcgcgaacaccagcatcaggccaagctgcgcgagcagaagctccacggcggcaccgtgttcaacccgaccctcgaataccttgaggtcaccctgcccttccactcgccgctcatggccgacgccgtcgagcagaccgtggcctgggccggcgcctgcggcttcgaccagaagcgcacccgtgcgcttgccgaggaagtgcttctgaaccatgtggactggaacgcccgcgtcaaggcgctgttcgacgccgccgacccgtccaagctgtggatcgtggacctcggccccggcaacacgctcggcaagctgatcggcaatgtggtgcaaggcaccggcatcggcgtggtcgaagccaccacgctggccgagcgctccacgctctccacgctggaaagcgagcctgagcgcacccagaactggaaggcattcgccccgcgcgtcatcaacaccccggccggcgccaagctggtcaccaagttctccaagctcaccggcaagccgccggtactgttgcccagcatgacccccaccaccgtggaaccggaaatcgtggccgccgcggccaacgccggctactgggccgagctcgccggcggcggccaggtgaccgccgaagtgttcgaccgccacatcgccaccctcgaagacgagctggaggaaggccgcacggtcgaattcaacgcgatgttcatggaccgttacctgtggaacctgcagttcggctcctcccgcatcgtgcccaagaagcgcgcatccggcgcgccgatcgacggcgtggtcgtctccgccggcatccccgagctggacgaagccgtggccctgatcaagtcgctgcaggcggacggcctgccgtacgtgagcttcaagcccggcaccgtggaccagattcgtcaggtcgtgcgcatcgccaaggccgtggcccccaccaccatcatggttcaggtcgagggcggtgaagccggcggccaccacagctgggaagcgctggatgacctgcttgccgccacctacgccgaagtgcgcgcctgtgacaacctcgtgctggtcgccggcggcggcatcggcactcccgaacgcgcggccgactacatttccggccagtgggcccacgcctacggcctgcccgacatgccggtcgacggcgtgcttatcggtaccgccgcgatgaccgccaaggaggctcacaccagccccgaagtcaagcagctgctcgtcaagacgcccggtattccggccgacgccaagtccgacgacgtgttcgccccgcaggccgcccactgggtgccgtccggcaagtccgtcggtggcatgtcctccggcctgagccacctgcacgccgacatttacgagctcgagaacgactccgccgaatgcggccgtctgctcgtgcgcgtcatgaagcaccccgaagagctcgactcccgccgcaaggaaatcatcgaagccctgaacaagaccgccaagccttacttcggcgacctggcctccatgacctaccttgagtgggcgcagcgcttcgccgaactcgccttcccgtgggtcgacccgacttacgccgaccgcttccagcacctgctgcagcgcatcgaagcccgtgtgaacgacacggactccggcgaattcacctccaagttgttcgccgctgatggcgtttctgctgaagaggcagctgccgctggcctgctgacccacgacgacattctcgccgacccggcccccgcgctcgaaaagctcgcactggcctacccgcagaccgccgaactcaaggtggtcccgaccgacgtggcttggttcccggtgctcgtgcgcgaataccccaagcccatgccgttcgtgcccgtcatcgacaacgacctgctgcgttggtgggggcaggaccagctctggcagtccgaagaccagcgctactccgccgattccgtgcgtgccatccccggcccgatctccgttgccggcatcaccaccatcgacgagccgatcgccgacatcctcggccgcttcgaaaccgccgccatcaagcgcgtgcaggacgaacagcaggccgccgatgccgctgagaacgacgatttcgccgcacttggcgaggccaccagcgccgaagacttcatccgcaagtccccgaacatctcctgggtcggccacatcaccgacaacccggcctacggcaccgccctgggcgaccagtactacgagatccgcgcgtttgacgccgcggccggcaagtacgatctggacatccacctcgacacctactgggacaacgacccggacggcggcacctccaagcacgccgtgcgcgatatcgtcatcccgctcatcgtggaaggcgccgagcccggccgtgtgccggtcgtggaccgcgaacgcctgattccggacgtctacgcgatgctcgccgccaccgcaggcatcggcaacaccgccatcaccggcgacaagctcaccgagatgccgcagcttgacggcaagacctctgacgagcgtccgtttgctaccgccaccgccgcctacaccctctccgccaacctcggcttcgaccacgaagcggccaccggcgccgctctgccgacgaccctgcagcccagccgcatcgctcccgatgcgctggtcggcccggcctggccggcgatttacacggcgctcggctccgtgtacgtcaagggctacccggtgattgaaggcctgctcaacgccgttcacctcgaccacctcatcgaacttgaggtgaacgaggaggacctgcttaagcacaccggtgaaaccatcaccctgaagtcttgggctgaggattacttcgagtccgcctccggccgcgtggtcaccatccacgtgacccacaccgctgccgacggcacgctgcttgcccgcgaaaccgagcgtttcgcgattcgcggccgcgtctactccgatgctttgccggcagacgcccccgagttcggcgacgccgagcacgaggagatcgcgcccacgccgcgccgtctgctgcgccgcgtgaaggtcgtcgccccacacgacatgaccgccttcgcccgcacttccggcgacttcaacccgatccacacctccactcgtggcgctcgcatctccggtttggccgctccgcttgtgcacggcatgtggctgtccgccaccgcccagcacgccgtgcaggcgctggacgagaagggtgcccactacgagattgccggctggacctacaacatgtacggcatggttcagcttgacgacaccgtcgaaatctccgtcgagcgcgtcggcaaggtgcgccacggcggcatgaccctcgaagtcacctgccgtatcgacggccagctcgtctcccgcggcaccgcactggtccgcgcgccgcgcgccgccttcgtctaccccggccagggcatccagaagcagggcatggttctggacgagcgtgccaagtccgccgccgcccgcgacgtgtgggagcgcgccgacaagctcacccgctccaagctcggcttctcgatcctgggcctcgtgcgcgataacccgaaggagctcaccgccaacggcgtgacctatcgccaccccgaaggcctgctcaacctgacgcagttcacgcaggtggcgctcgccaccgtggccttcgcccagaccgctcgtctgcgcgaagccggcgccgacatctggcccgcctacttcgccggccactcgctcggcgaatacaacgcgctgtccgcgttcgccgacgtgattccgctggaaaccgtgctggagctcgtgttccaccgtggttccaccatgcaccacctcatcgagcgtgacgcacagggtcgctccaactaccgcatgggtgccctgcgcccgaaccagttcggtgtggacgacgcgcacgtcaaggaatacgtggaatccgtggccaaggcgtccggcgaattcctggagatcgtgaactacaacctcgccggccagcagtacgccatcgccggtacgatcgccggactcaaggctctgaaggccgattccgcccgccgcgtggccgcattcggtggcaagcccgcgttcatgctggtgcccggcatcgatgtgccgttccactccacactgctgcgcaagggcgtgccggaattccgcgacaagctcgacgcgctgcttcccgcttacatcgactaccgtggccgtctggtggaccgctacatcccgaacctggtggccaccccgttcgagatgaccaaggaattcgccgccaagatcctcgaagtcgtgccgtccgagcgtatcaaggccgtgctcgacgacccggccgtgtgggattcctacgccgatgacgaccagaagctcggccgtctgctcttgaccgaactgctctcctggcagttcgcctccccggtgcgttggattgaaacccaggcgctgctcttcggtcagcgcgaacagggcggcctcggcgtggaggaatacgtcgaagtcggcctcggcaacgccccgaccctggccaacctcggtgccaagaccctgcgtctacccgagttcgccggcaacgatacggtcgtctacaacgtcggccgcgatgaaggccgcgtctacatgaccgacaccgattcgctggttcccgacgatgagccggaagaggctgcggcctcggccccggtcgaaaccgctcctgccggtggttccgccgtcgccaagctcggttccggtgccgctccggcaaatgccgtgactgaggctgctcagacaacggatggctccgtcgccggacagtccactggactgtccgactcctcgcgcggcctgcaatcagggcaggccgcggccggcgcaccgtccggtgccgccgtcgccgacctgcccttcaaggcttccgatgccatcggcgtgctcatggcctactccgccaaggtgcgcatcgaccagatcggcagcaacgacaccactgacaccctgaccaacggtgtctcttctcgccgcaaccagctgctcatggacatcagctccgagctgggagtcgccagcgtggacggtgctgctgaagcgacgctcgacaagctcgcccaaatcgtcaacaaggccgccccgaactacaagccgttcggtgccgtgctctccgaggcgctgcgcgaccgcctgcgctcgctgttcggtgccgccggcgtcaagcagcagtacatccgcgaccgcgtgaccaacgtatggcagctcggtgaaggttgggtggccagcgtgctcgccgcactgctgctcgacactcgcgagggtgccagctcccgtggcggcgacctggccaagctgcccaccgccgccgtgcagaacaagccggaagccgacaagctgattgatgcggccgttgaggtcgtcgcccagctcaagggtgttgccgtggccctgccgtccgccggtggtgccgctggtggcgctgtggtcgattccgccgcgctcgacgccttcgccgagaaggtcaccggttccaacggcgtgctcgccgccaccgcccgcttcgtgctgaacgagctcggcgtggccgctccggctccggaagaatccgaggacgagaacgccgccgtcgtggccgcggttgaagccgaacttggctccgactggcctaagcaggtcgctccgcgcttcgacgccaacaaggccatcctcttcgatgatcggtgggcctccgcccgtgaagacctcgcccgcgcctactacgacaacgatcccgccgccttgaacggcagcttcatcggccttggcaagaccatcgccgccgaagcccagtggttcgccaacgagtcggagagcgaagaactcaaggccgccttccagaaggccggctccgaggccctcgaacaggttgcgtccaacaagaacgcctcccgttacgcgaatgatatcgccatcgtcaccggcgtgagcccgaactcgattgccgctcaggtggtcgaaggcctgctcgcaggcggtgccactgtggtggccacctcgcacagcttcaagccgtccattaaggcttgggctaagcaggcctaccgcgagcacgctaccggcaacgccaagctgtggctggttccggccaacctctccagctaccgcgacgtcgacgcactggtcgactgggtgggccacgagcagaagaagacctccggtgctaccaccacgattctgaagcccgcgtgggagccgactctgttcttcccgttcgccgctccgccggtccatggcacgctcgccgactccggcgacctgttcgaatcgcaggcccgactgatgctgtggggcgtggaacgcgccatcgccggcttcagccacatcggtgccgacaccaacgtgcagcacaagctccacgtggtgctgcccggttccccgaaccgcggcgtattcggcggcgacggtgcctacggcgaagtcaagagcgcctttgacgccatcgtcaaccgtgcccgcgctgaaaaggtgtggtcgagccgcgtcaccttcgctcacccgaagatcggctgggtgcgtggcaccggcctgatggtcggtaacgacccgctggtcgccgtcgttgagcgtcacggcatccgcacctactccaccgcacagattgcggccaagctgctcgacctgtgcactgccgaatcgcgcgagcaggccctcaaggccccgctcgatgtggacctgaccggtggcctcggctccgagcccatcgacatcaaggccctgcgtgcggaggccatggccgacgccgagaaggaagctgctgcggcctcgtctcaggaaacggatggctccgtcgccggaaagtccactggactgtccgactcctcgcgtgggcagcaaatcaaggcgctgcccacaccgatcgtcaccaagcaggctccggtcgacttgaacgattggaccaacgtcactgccaagccggaagacgaaatcgtcatcgtttccgtcggtgagctgggcccgtggggctccggtcgtacgcgcgcccaggccgagctcggcatccactcggacggcaccgtcgacctcagcgccggcgcagtgctcgaactcgcgtggaacatgggcctgctgacttgggccgacagcccgaagcccggctggtacgacaccgacggcaacctcgtgcccgaagaggacatcgccgagcgctaccacgacgaggtcgtggcccgctccggcatccgcccgttcgaggaaggcatgggcaacgactacaaggacggtgctgacgaagaggaagccgaggtcttcctcgaccacgacgtcaccttctctgtgcccacccgcgaagtcgccgccgaatacgtcaagttggacgaggctcacaccaccatcgccccggatgaggaatccggcgaatggaacgtgacccgccacgccggctcgatgatccgcgtgccgcgccgcgccaccatgacccgcacagtcggcggccagttcccgaagggcttcgacccgacccgatggggcattcccgcctcgatggtcggcgacgtcgacaagatcgccctgtggaacatcgtcaccactgtggacgcctacctgggcgccggcttcaccccggccgaaatcctcgagtccatccacccctcgctggtggcttcgacccagggcaccggcttcggtggcatgatgtcgatgcgcaagctgtacctcgaccgcttcctcaaccacgagatcccgaccgacatcctgcaggaagccctgccgaacgtggtcgcggctcacgtgatgcagtcctacatcggcggctacggcaacatgattcagccggtctccgcctgcgccaccgcagccgtctcccttgaagagggcgtcgacaagatcgccctcggcaaggccgacttcgtggtcaccggtgcgatcgacgacatcggcgtggaatccgtgatcggtttcggcaacatgaacgccaccgccaactcggaggaaatgtatggcaagggtatcgacgcgcgcttcttcagccgcgcgaacgatcgtcgccgtggcggcttcctggaatcccagggcggcggcacgatcctcgtgacccgcggtgacatcgccgaaaagctcggcctgccggttgcggccgtggtcggcttcatccactcctacgcggatggcgctcacacctcgatcccggctccgggcctcggtgccctggctgccggcctcggcggcaaggactccaagctcgtgcacgacctggctaagctcggcgtctcagccgacgacatcgccgtggtctccaagcacgacacgtccaccaacgccaacgacccgaacgaatccgagctgcacaacacgctggcccacgccatcggccgcaccgacggcaacccgctgttcgtgatctcgcagaagacgcttaccggccacgccaagggtggtgcctgcatcttccaggtcaacggtctgacgcagctgttcaagtccggcgtgattccggcgaacgcggcgctcgactgcgtggatccgaagctccagcgcgacgaccacatggtctgggttaggaagcccctgcgcatcggcggtggtgaggacgagttcggtcgcgagaccgctggtcgcccggtcaaggccggtctggccacctcgctcggcttcggacatgtttccggcttcgtggctctggtccatccgggtgccttcgaggcggccgtggccaaggccgatggtgaggccgcactggaagcttggcgtgagcgtgccaacgcccgcctggctgccggccagcgtcaccttgaggaaggcatgatgggtcgcgccgcgctctacgagccgatcgacaaccgtcgcttccgcgaggatcaccgtggctacgaccaccacgaggtcgaaaaggcgatgctcctcaacccggacgcccgcctcggtgccgacggctactacgaggcctga

SEQ ID NO: 2 - protein, 3172 aa, synthetic construct
MTTFFLNRLATEPHALAFAGQSTPWPVALADQTTDPSLDEALRGHIAASNRLLTPVAADLLTTTGRPVDLFGFTPNPARLGAAADATASVEGVALTQLGALLDLEHLGYSVAQAKPLAVLGHSQGVLAVHMARAIEAAGSIEAAGKTLDEILAVAALIGAAGTRRVRELGLNPKYGEASPMLSVKGATREQVEALVARVNNARGPISIAVTNSDNHHVLSGYPEDLSAFALEAEREHQHQAKLREQKLHGGTVFNPTLEYLEVTLPFHSPLMADAVEQTVAWAGACGFDQKRTRALAEEVLLNHVDWNARVKALFDAADPSKLWIVDLGPGNTLGKLIGNVVQGTGIGVVEATTLAERSTLSTLESEPERTQNWKAFAPRVINTPAGAKLVTKFSKLTGKPPVLLPSMTPTTVEPEIVAAAANAGYWAELAGGGQVTAEVFDRHIATLEDELEEGRTVEFNAMFMDRYLWNLQFGSSRIVPKKRASGAPIDGVVVSAGIPELDEAVALIKSLQADGLPYVSFKPGTVDQIRQVVRIAKAVAPTTIMVQVEGGEAGGHHSWEALDDLLAATYAEVRACDNLVLVAGGGIGTPERAADYISGQWAHAYGLPDMPVDGVLIGTAAMTAKEAHTSPEVKQLLVKTPGIPADAKSDDVFAPQAAHWVPSGKSVGGMSSGLSHLHADIYELENDSAECGRLLVRVMKHPEELDSRRKEIIEALNKTAKPYFGDLASMTYLEWAQRFAELAFPWVDPTYADRFQHLLQRIEARVNDTDSGEFTSKLFAADGVSAEEAAAAGLLTHDDILADPAPALEKLALAYPQTAELKVVPTDVAWFPVLVREYPKPMPFVPVIDNDLLRWWGQDQLWQSEDQRYSADSVRAIPGPISVAGITTIDEPIADILGRFETAAIKRVQDEQQAADAAENDDFAALGEATSAEDFIRKSPNISWVGHITDNPAYGTALGDQYYEIRAFDAAAGKYDLDIHLDTYWDNDPDGGTSKHAVRDIVIPLIVEGAEPGRVPVVDRERLIPDVYAMLAATAGIGNTAITGDKLTEMPQLDGKTSDERPFATATAAYTLSANLGFDHEAATGAALPTTLQPSRIAPDALVGPAWPAIYTALGSVYVKGYPVIEGLLNAVHLDHLIELEVNEEDLLKHTGETITLKSWAEDYFESASGRVVTIHVTHTAADGTLLARETERFAIRGRVYSDALPADAPEFGDAEHEEIAPTPRRLLRRVKVVAPHDMTAFARTSGDFNPIHTSTRGARISGLAAPLVHGMWLSATAQHAVQALDEKGAHYEIAGWTYNMYGMVQLDDTVEISVERVGKVRHGGMTLEVTCRIDGQLVSRGTALVRAPRAAFVYPGQGIQKQGMVLDERAKSAAARDVWERADKLTRSKLGFSILGLVRDNPKELTANGVTYRHPEGLLNLTQFTQVALATVAFAQTARLREAGADIWPAYFAGHSLGEYNALSAFADVIPLETVLELVFHRGSTMHHLIERDAQGRSNYRMGALRPNQFGVDDAHVKEYVESVAKASGEFLEIVNYNLAGQQYAIAGTIAGLKALKADSARRVAAFGGKPAFMLVPGIDVPFHSTLLRKGVPEFRDKLDALLPAYIDYRGRLVDRYIPNLVATPFEMTKEFAAKILEVVPSERIKAVLDDPAVWDSYADDDQKLGRLLLTELLSWQFASPVRWIETQALLFGQREQGGLGVEEYVEVGLGNAPTLANLGAKTLRLPEFAGNDTVVYNVGRDEGRVYMTDTDSLVPDDEPEEAAASAPVETAPAGGSAVAKLGSGAAPANAVTEAAQTTDGSVAGQSTGLSDSSRGLQSGQAAAGAPSGAAVADLPFKASDAIGVLMAYSAKVRIDQIGSNDTTDTLTNGVSSRRNQLLMDISSELGVASVDGAAEATLDKLAQIVNKAAPNYKPFGAVLSEALRDRLRSLFGAAGVKQQYIRDRVTNVWQLGEGWVASVLAALLLDTREGASSRGGDLAKLPTAAVQNKPEADKLIDAAVEVVAQLKGVAVALPSAGGAAGGAVVDSAALDAFAEKVTGSNGVLAATARFVLNELGVAAPAPEESEDENAAVVAAVEAELGSDWPKQVAPRFDANKAILFDDRWASAREDLARAYYDNDPAALNGSFIGLGKTIAAEAQWFANESESEELKAAFQKAGSEALEQVASNKNASRYANDIAIVTGVSPNSIAAQVVEGLLAGGATVVATSHSFKPSIKAWAKQAYREHATGNAKLWLVPANLSSYRDVDALVDWVGHEQKKTSGATTTILKPAWEPTLFFPFAAPPVHGTLADSGDLFESQARLMLWGVERAIAGFSHIGADTNVQHKLHVVLPGSPNRGVFGGDGAYGEVKSAFDAIVNRARAEKVWSSRVTFAHPKIGWVRGTGLMVGNDPLVAVVERHGIRTYSTAQIAAKLLDLCTAESREQALKAPLDVDLTGGLGSEPIDIKALRAEAMADAEKEAAAASSQETDGSVAGKSTGLSDSSRGQQIKALPTPIVTKQAPVDLNDWTNVTAKPEDEIVIVSVGELGPWGSGRTRAQAELGIHSDGTVDLSAGAVLELAWNMGLLTWADSPKPGWYDTDGNLVPEEDIAERYHDEVVARSGIRPFEEGMGNDYKDGADEEEAEVFLDHDVTFSVPTREVAAEYVKLDEAHTTIAPDEESGEWNVTRHAGSMIRVPRRATMTRTVGGQFPKGFDPTRWGIPASMVGDVDKIALWNIVTTVDAYLGAGFTPAEILESIHPSLVASTQGTGFGGMMSMRKLYLDRFLNHEIPTDILQEALPNVVAAHVMQSYIGGYGNMIQPVSACATAAVSLEEGVDKIALGKADFVVTGAIDDIGVESVIGFGNMNATANSEEMYGKGIDARFFSRANDRRRGGFLESQGGGTILVTRGDIAEKLGLPVAAVVGFIHSYADGAHTSIPAPGLGALAAGLGGKDSKLVHDLAKLGVSADDIAVVSKHDTSTNANDPNESELHNTLAHAIGRTDGNPLFVISQKTLTGHAKGGACIFQVNGLTQLFKSGVIPANAALDCVDPKLQRDDHMVWVRKPLRIGGGEDEFGRETAGRPVKAGLATSLGFGHVSGFVALVHPGAFEAAVAKADGEAALEAWRERANARLAAGQRHLEEGMMGRAALYEPIDNRRFREDHRGYDHHEVEKAMLLNPDARLGADGYYEA

SEQ ID NO: 3 - DNA, 9519 bp, Bifidobacterium longum
  source 1..9519: mol_type = genomic DNA
atgaccaccttcttcctcaatcgcctagccaccgagccccatgcgctcgccttcgccggccagtccaccccgtggccggtagccctggcagaccagaccaccgatcccagccttgacgaggccctgcgcggccacatcgccgcatccaaccgactgctcaccccggtcgccgccgacctgctgaccaccaccggccgtccggtcgacctgttcggcttcactccgaacccggcccgcctcggcgccgcagccgacgccaccgcgtccgtggaaggcgtggcactgacccagctcggcgccctgctcgacctcgaacacctcggctacagcgtggcccaggccaagccgctcgccgtgctcggccactcccagggcgtgctcgccgtgcacatggcccgcgccatcgaggccgccggctccatcgaggccgccggcaagacccttgacgagattctggccgtcgccgccctgatcggtgcagccggcactcgccgcgtgcgcgaactgggcctgaaccccaagtatggcgaagccagtccgatgctctccgtcaagggcgccacccgcgaacaggttgaggctctggtggcccgcgtcaacaacgctcgcggcccgatctccatcgccgtcaccaactccgacaaccaccacgtgctttccggctacccggaggacctgtctgccttcgccctcgaagccgagcgcgaacaccagcatcaggccaagctgcgcgagcagaagctccacggcggcaccgtgttcaacccgaccctcgaataccttgaggtcaccctgcccttccactcgccgctcatggccgacgccgtcgagcagaccgtggcctgggccggcgcctgcggcttcgaccagaagcgcacccgtgcgcttgccgaggaagtgcttctgaaccatgtggactggaacgcccgcgtcaaggcgctgttcgacgccgccgacccgtccaagctgtggatcgtggacctcggccccggcaacacgctcggcaagctgatcggcaatgtggtgcaaggcaccggcatcggcgtggtcgaagccaccacgctggccgagcgctccacgctctccacgctggaaagcgagcctgagcgcacccagaactggaaggcattcgccccgcgcgtcatcaacaccccggccggcgccaagctggtcaccaagttctccaagctcaccggcaagccgccggtactgttgccccgcatgacccccaccaccgtggaaccggaaatcgtggccgccgcggccaacgccggctactgggccgagctcgccggcggcggccaggtgaccgccgaagtgttcgaccgccacatcgccaccctcgaagacgagctggaggaaggccgcacggtcgaattcaacgcgatgttcatggaccgttacctgtggaacctgcagttcggctcctcccgcatcgtgcccaagaagcgcgcatccggcgcgccgatcgacggcgtggtcgtctccgccggcatccccgagctggacgaagccgtggccctgatcaagtcgctgcaggcggacggcctgccgtacgtgagcttcaagcccggcaccgtggaccagattcgtcaggtcgtgcgcatcgccaaggccgtggcccccaccaccatcatggttcaggtcgagggcggtgaagccggcggccaccacagctgggaagcgctggatgacctgcttgccgccacctacgccgaagtgcgcgcctgtgacaacctcgtgctggtcgccggcggcggcatcggcactcccgaacgcgcggccgactacatttccggccagtgggcccacgcctacggcctgcccgacatgccggtcgacggcgtgcttatcggtaccgccgcgatgaccgccaaggaggctcacaccagccccgaagtcaagcagctgctcgtcaagacgcccggtattccggccgacgccaagtccgacgacgtgttcgccccgcaggccgcccactgggtgccgtccggcaagtccgtcggtggcatgtcctccggcctgagccacctgcacgccgacatttacgagctcgagaacgactccgccgaatgcggccgtctgctcgtgcgcgtcatgaagcaccccgaagagctcgactcccgccgcaaggaaatcatcgaagccctgaacaagaccgccaagccttacttcggcgacctggcctccatgacctaccttgagtgggcgcagcgcttcgccgaactcgccttcccgtgggtcgacccgacttacgccgaccgcttccagcacctgctgcagcgcatcgaagcccgtgtgaacgacacggactccggcgaattcacctccaagttgttcgccgctgatggcgtttctgctgaagaggcagctgccgctggcctgctgacccacgacgacattctcgccgacccggcccccgcgctcgaaaagctcgcactggcctacccgcagaccgccgaactcaaggtggtcccgaccgacgtggcttggttcccggtgctcgtgcgcgaataccccaagcccatgccgttcgtgcccgtcatcgacaacgacctgctgcgttggtgggggcaggaccagctctggcagtccgaagaccagcgctactccgccgattccgtgcgtgccatccccggcccgatctccgttgccggcatcaccaccatcgacgagccgatcgccgacatcctcggccgcttcgaaaccgccgccatcaagcgcgtgcaggacgaacagcaggccgccgatgccgctgagaacgacgatttcgccgcacttggcgaggccaccagcgccgaagacttcatccgcaagtccccgaacatctcctgggtcggccacatcaccgacaacccggcctacggcaccgccctgggcgaccagtactacgagatccgcgcgtttgacgccgcggccggcaagtacgatctggacatccacctcgacacctactgggacaacgacccggacggcggcacctccaagcacgccgtgcgcgatatcgtcatcccgctcatcgtggaaggcgccgagcccggccgtgtgccggtcgtggaccgcgaacgcctgattccggacgtctacgcgatgctcgccgccaccgcaggcatcggcaacaccgccatcaccggcgacaagctcaccgagatgccgcagcttgacggcaagacctctgacgagcgtccgtttgctaccgccaccgccgcctacaccctctccgccaacctcggcttcgaccacgaagcggccaccggcgccgctctgccgacgaccctgcagcccagccgcatcgctcccgatgcgctggtcggcccggcctggccggcgatttacacggcgctcggctccgtgtacgtcaagggctacccggtgattgaaggcctgctcaacgccgttcacctcgaccacctcatcgaacttgaggtgaacgaggaggacctgcttaagcacaccggtgaaaccatcaccctgaagtcttgggctgaggattacttcgagtccgcctccggccgcgtggtcaccatccacgtgacccacaccgctgccgacggcacgctgcttgcccgcgaaaccgagcgtttcgcgattcgcggccgcgtctactccgatgctttgccggcagacgcccccgagttcggcgacgccgagcacgaggagatcgcgcccacgccgcgccgtctgctgcgccgcgtgaaggtcgtcgccccacacgacatgaccgccttcgcccgcacttccggcgacttcaacccgatccacacctccactcgtggcgctcgcatctccggtttggccgctccgcttgtgcacggcatgtggctgtccgccaccgcccagcacgccgtgcaggcgctggacgagaagggtgcccactacgagattgccggctggacctacaacatgtacggcatggttcagcttgacgacaccgtcgaaatctccgtcgagcgcgtcggcaaggtgcgccacggcggcatgaccctcgaagtcacctgccgtatcgacggccagctcgtctcccgcggcaccgcactggtccgcgcgccgcgcgccgccttcgtctaccccggccagggcatccagaagcagggcatggttctggacgagcgtgccaagtccgccgccgcccgcgacgtgtgggagcgcgccgacaagctcacccgctccaagctcggcttctcgatcctgggcctcgtgcgcgataacccgaaggagctcaccgccaacggcgtgacctatcgccaccccgaaggcctgctcaacctgacgcagttcacgcaggtggcgctcgccaccgtggccttcgcccagaccgctcgtctgcgcgaagccggcgccgacatctggcccgcctacttcgccggccactcgctcggcgaatacaacgcgctgtccgcgttcgccgacgtgattccgctggaaaccgtgctggagctcgtgttccaccgtggttccaccatgcaccacctcatcgagcgtgacgcacagggtcgctccaactaccgcatgggtgccctgcgcccgaaccagttcggtgtggacgacgcgcacgtcaaggaatacgtggaatccgtggccaaggcgtccggcgaattcctggagatcgtgaactacaacctcgccggccagcagtacgccatcgccggtacgatcgccggactcaaggctctgaaggccgattccgcccgccgcgtggccgcattcggtggcaagcccgcgttcatgctggtgcccggcatcgatgtgccgttccactccacactgctgcgcaagggcgtgccggaattccgcgacaagctcgacgcgctgcttcccgcttacatcgactaccgtggccgtctggtggaccgctacatcccgaacctggtggccaccccgttcgagatgaccaaggaattcgccgccaagatcctcgaagtcgtgccgtccgagcgtatcaaggccgtgctcgacgacccggccgtgtgggattcctacgccgatgacgaccagaagctcggccgtctgctcttgaccgaactgctctcctggcagttcgcctccccggtgcgttggattgaaacccaggcgctgctcttcggtcagcgcgaacagggcggcctcggcgtggaggaatacgtcgaagtcggcctcggcaacgccccgaccctggccaacctcggtgccaagaccctgcgtctacccgagttcgccggcaacgatacggtcgtctacaacgtcggccgcgatgaaggccgcgtctacatgaccgacaccgattcgctggttcccgacgatgagccggaagaggctgcggcctcggccccggtcgaaaccgctcctgccggtggttccgccgtcgccaagctcggttccggtgccgctccggcaaatgccgtgactgaggctgctcagacaacggatggctccgtcgccggacagtccactggactgtccgactcctcgcgcggcctgcaatcagggcaggccgcggccggcgcaccgtccggtgccgccgtcgccgacctgcccttcaaggcttccgatgccatcggcgtgctcatggcctactccgccaaggtgcgcatcgaccagatcggcagcaacgacaccactgacaccctgaccaacggtgtctcttctcgccgcaaccagctgctcatggacatcagctccgagctgggagtcgccagcgtggacggtgctgctgaagcgacgctcgacaagctcgcccaaatcgtcaacaaggccgccccgaactacaagccgttcggtgccgtgctctccgaggcgctgcgcgaccgcctgcgctcgctgttcggtgccgccggcgtcaagcagcagtacatccgcgaccgcgtgaccaacgtatggcagctcggtgaaggttgggtggccagcgtgctcgccgcactgctgctcgacactcgcgagggtgccagctcccgtggcggcgacctggccaagctgcccaccgccgccgtgcagaacaagccggaagccgacaagctgattgatgcggccgttgaggtcgtcgcccagctcaagggtgttgccgtggccctgccgtccgccggtggtgccgctggtggcgctgtggtcgattccgccgcgctcgacgccttcgccgagaaggtcaccggttccaacggcgtgctcgccgccaccgcccgcttcgtgctgaacgagctcggcgtggccgctccggctccggaagaatccgaggacgagaacgccgccgtcgtggccgcggttgaagccgaacttggctccgactggcctaagcaggtcgctccgcgcttcgacgccaacaaggccatcctcttcgatgatcggtgggcctccgcccgtgaagacctcgcccgcgcctactacgacaacgatcccgccgccttgaacggcagcttcatcggccttggcaagaccatcgccgccgaagcccagtggttcgccaacgagtcggagagcgaagaactcaaggccgccttccagaaggccggctccgaggccctcgaacaggttgcgtccaacaagaacgcctcccgttacgcgaatgatatcgccatcgtcaccggcgtgagcccgaactcgattgccgctcaggtggtcgaaggcctgctcgcaggcggtgccactgtggtggccacctcgcacagcttcaagccgtccattaaggcttgggctaagcaggcctaccgcgagcacgctaccggcaacgccaagctgtggctggttccggccaacctctccagctaccgcgacgtcgacgcactggtcgactgggtgggccacgagcagaagaagacctccggtgctaccaccacgattctgaagcccgcgtgggagccgactctgttcttcccgttcgccgctccgccggtccatggcacgctcgccgactccggcgacctgttcgaatcgcaggcccgactgatgctgtggggcgtggaacgcgccatcgccggcttcagccacatcggtgccgacaccaacgtgcagcacaagctccacgtggtgctgcccggttccccgaaccgcggcgtattcggcggcgacggtgcctacggcgaagtcaagagcgcctttgacgccatcgtcaaccgtgcccgcgctgaaaaggtgtggtcgagccgcgtcaccttcgctcacccgaagatcggctgggtgcgtggcaccggcctgatggtcggtaacgacccgctggtcgccgtcgttgagcgtcacggcatccgcacctactccaccgcacagattgcggccaagctgctcgacctgtgcactgccgaatcgcgcgagcaggccctcaaggccccgctcgatgtggacctgaccggtggcctcggctccgagcccatcgacatcaaggccctgcgtgcggaggccatggccgacgccgagaaggaagctgctgcggcctcgtctcaggaaacggatggctccgtcgccggaaagtccactggactgtccgactcctcgcgtgggcagcaaatcaaggcgctgcccacaccgatcgtcaccaagcaggctccggtcgacttgaacgattggaccaacgtcactgccaagccggaagacgaaatcgtcatcgtttccgtcggtgagctgggcccgtggggctccggtcgtacgcgcgcccaggccgagctcggcatccactcggacggcaccgtcgacctcagcgccggcgcagtgctcgaactcgcgtggaacatgggcctgctgacttgggccgacagcccgaagcccggctggtacgacaccgacggcaacctcgtgcccgaagaggacatcgccgagcgctaccacgacgaggtcgtggcccgctccggcatccgcccgttcgaggaaggcatgggcaacgactacaaggacggtgctgacgaagaggaagccgaggtcttcctcgaccacgacgtcaccttctctgtgcccacccgcgaagtcgccgccgaatacgtcaagttggacgaggctcacaccaccatcgccccggatgaggaatccggcgaatggaacgtgacccgccacgccggctcgatgatccgcgtgccgcgccgcgccaccatgacccgcacagtcggcggccagttcccgaagggcttcgacccgacccgatggggcattcccgcctcgatggtcggcgacgtcgacaagatcgccctgtggaacatcgtcaccactgtggacgcctacctgggcgccggcttcaccccggccgaaatcctcgagtccatccacccctcgctggtggcttcgacccagggcaccggcttcggtggcatgatgtcgatgcgcaagctgtacctcgaccgcttcctcaaccacgagatcccgaccgacatcctgcaggaagccctgccgaacgtggtcgcggctcacgtgatgcagtcctacatcggcggctacggcaacatgattcagccggtctccgcctgcgccaccgcagccgtctcccttgaagagggcgtcgacaagatcgccctcggcaaggccgacttcgtggtcaccggtgcgatcgacgacatcggcgtggaatccgtgatcggtttcggcaacatgaacgccaccgccaactcggaggaaatgtatggcaagggtatcgacgcgcgcttcttcagccgcgcgaacgatcgtcgccgtggcggcttcctggaatcccagggcggcggcacgatcctcgtgacccgcggtgacatcgccgaaaagctcggcctgccggttgcggccgtggtcggcttcatccactcctacgcggatggcgctcacacctcgatcccggctccgggcctcggtgccctggctgccggcctcggcggcaaggactccaagctcgtgcacgacctggctaagctcggcgtctcagccgacgacatcgccgtggtctccaagcacgacacgtccaccaacgccaacgacccgaacgaatccgagctgcacaacacgctggcccacgccatcggccgcaccgacggcaacccgctgttcgtgatctcgcagaagacgcttaccggccacgccaagggtggtgcctgcatcttccaggtcaacggtctgacgcagctgttcaagtccggcgtgattccggcgaacgcggcgctcgactgcgtggatccgaagctccagcgcgacgaccacatggtctgggttaggaagcccctgcgcatcggcggtggtgaggacgagttcggtcgcgagaccgctggtcgcccggtcaaggccggtctggccacctcgctcggcttcggacatgtttccggcttcgtggctctggtccatccgggtgccttcgaggcggccgtggccaaggccgatggtgaggccgcactggaagcttggcgtgagcgtgccaacgcccgcctggctgccggccagcgtcaccttgaggaaggcatgatgggtcgcgccgcgctctacgagccgatcgacaaccgtcgcttccgcgaggatcaccgtggctacgaccaccacgaggtcgaaaaggcgatgctcctcaacccggacgcccgcctcggtgccgacggctactacgaggcctga

SEQ ID NO: 4 - protein, 3172 aa, Bifidobacterium longum
MTTFFLNRLATEPHALAFAGQSTPWPVALADQTTDPSLDEALRGHIAASNRLLTPVAADLLTTTGRPVDLFGFTPNPARLGAAADATASVEGVALTQLGALLDLEHLGYSVAQAKPLAVLGHSQGVLAVHMARAIEAAGSIEAAGKTLDEILAVAALIGAAGTRRVRELGLNPKYGEASPMLSVKGATREQVEALVARVNNARGPISIAVTNSDNHHVLSGYPEDLSAFALEAEREHQHQAKLREQKLHGGTVFNPTLEYLEVTLPFHSPLMADAVEQTVAWAGACGFDQKRTRALAEEVLLNHVDWNARVKALFDAADPSKLWIVDLGPGNTLGKLIGNVVQGTGIGVVEATTLAERSTLSTLESEPERTQNWKAFAPRVINTPAGAKLVTKFSKLTGKPPVLLPRMTPTTVEPEIVAAAANAGYWAELAGGGQVTAEVFDRHIATLEDELEEGRTVEFNAMFMDRYLWNLQFGSSRIVPKKRASGAPIDGVVVSAGIPELDEAVALIKSLQADGLPYVSFKPGTVDQIRQVVRIAKAVAPTTIMVQVEGGEAGGHHSWEALDDLLAATYAEVRACDNLVLVAGGGIGTPERAADYISGQWAHAYGLPDMPVDGVLIGTAAMTAKEAHTSPEVKQLLVKTPGIPADAKSDDVFAPQAAHWVPSGKSVGGMSSGLSHLHADIYELENDSAECGRLLVRVMKHPEELDSRRKEIIEALNKTAKPYFGDLASMTYLEWAQRFAELAFPWVDPTYADRFQHLLQRIEARVNDTDSGEFTSKLFAADGVSAEEAAAAGLLTHDDILADPAPALEKLALAYPQTAELKVVPTDVAWFPVLVREYPKPMPFVPVIDNDLLRWWGQDQLWQSEDQRYSADSVRAIPGPISVAGITTIDEPIADILGRFETAAIKRVQDEQQAADAAENDDFAALGEATSAEDFIRKSPNISWVGHITDNPAYGTALGDQYYEIRAFDAAAGKYDLDIHLDTYWDNDPDGGTSKHAVRDIVIPLIVEGAEPGRVPVVDRERLIPDVYAMLAATAGIGNTAITGDKLTEMPQLDGKTSDERPFATATAAYTLSANLGFDHEAATGAALPTTLQPSRIAPDALVGPAWPAIYTALGSVYVKGYPVIEGLLNAVHLDHLIELEVNEEDLLKHTGETITLKSWAEDYFESASGRVVTIHVTHTAADGTLLARETERFAIRGRVYSDALPADAPEFGDAEHEEIAPTPRRLLRRVKVVAPHDMTAFARTSGDFNPIHTSTRGARISGLAAPLVHGMWLSATAQHAVQALDEKGAHYEIAGWTYNMYGMVQLDDTVEISVERVGKVRHGGMTLEVTCRIDGQLVSRGTALVRAPRAAFVYPGQGIQKQGMVLDERAKSAAARDVWERADKLTRSKLGFSILGLVRDNPKELTANGVTYRHPEGLLNLTQFTQVALATVAFAQTARLREAGADIWPAYFAGHSLGEYNALSAFADVIPLETVLELVFHRGSTMHHLIERDAQGRSNYRMGALRPNQFGVDDAHVKEYVESVAKASGEFLEIVNYNLAGQQYAIAGTIAGLKALKADSARRVAAFGGKPAFMLVPGIDVPFHSTLLRKGVPEFRDKLDALLPAYIDYRGRLVDRYIPNLVATPFEMTKEFAAKILEVVPSERIKAVLDDPAVWDSYADDDQKLGRLLLTELLSWQFASPVRWIETQALLFGQREQGGLGVEEYVEVGLGNAPTLANLGAKTLRLPEFAGNDTVVYNVGRDEGRVYMTDTDSLVPDDEPEEAAASAPVETAPAGGSAVAKLGSGAAPANAVTEAAQTTDGSVAGQSTGLSDSSRGLQSGQAAAGAPSGAAVADLPFKASDAIGVLMAYSAKVRIDQIGSNDTTDTLTNGVSSRRNQLLMDISSELGVASVDGAAEATLDKLAQIVNKAAPNYKPFGAVLSEALRDRLRSLFGAAGVKQQYIRDRVTNVWQLGEGWVASVLAALLLDTREGASSRGGDLAKLPTAAVQNKPEADKLIDAAVEVVAQLKGVAVALPSAGGAAGGAVVDSAALDAFAEKVTGSNGVLAATARFVLNELGVAAPAPEESEDENAAVVAAVEAELGSDWPKQVAPRFDANKAILFDDRWASAREDLARAYYDNDPAALNGSFIGLGKTIAAEAQWFANESESEELKAAFQKAGSEALEQVASNKNASRYANDIAIVTGVSPNSIAAQVVEGLLAGGATVVATSHSFKPSIKAWAKQAYREHATGNAKLWLVPANLSSYRDVDALVDWVGHEQKKTSGATTTILKPAWEPTLFFPFAAPPVHGTLADSGDLFESQARLMLWGVERAIAGFSHIGADTNVQHKLHVVLPGSPNRGVFGGDGAYGEVKSAFDAIVNRARAEKVWSSRVTFAHPKIGWVRGTGLMVGNDPLVAVVERHGIRTYSTAQIAAKLLDLCTAESREQALKAPLDVDLTGGLGSEPIDIKALRAEAMADAEKEAAAASSQETDGSVAGKSTGLSDSSRGQQIKALPTPIVTKQAPVDLNDWTNVTAKPEDEIVIVSVGELGPWGSGRTRAQAELGIHSDGTVDLSAGAVLELAWNMGLLTWADSPKPGWYDTDGNLVPEEDIAERYHDEVVARSGIRPFEEGMGNDYKDGADEEEAEVFLDHDVTFSVPTREVAAEYVKLDEAHTTIAPDEESGEWNVTRHAGSMIRVPRRATMTRTVGGQFPKGFDPTRWGIPASMVGDVDKIALWNIVTTVDAYLGAGFTPAEILESIHPSLVASTQGTGFGGMMSMRKLYLDRFLNHEIPTDILQEALPNVVAAHVMQSYIGGYGNMIQPVSACATAAVSLEEGVDKIALGKADFVVTGAIDDIGVESVIGFGNMNATANSEEMYGKGIDARFFSRANDRRRGGFLESQGGGTILVTRGDIAEKLGLPVAAVVGFIHSYADGAHTSIPAPGLGALAAGLGGKDSKLVHDLAKLGVSADDIAVVSKHDTSTNANDPNESELHNTLAHAIGRTDGNPLFVISQKTLTGHAKGGACIFQVNGLTQLFKSGVIPANAALDCVDPKLQRDDHMVWVRKPLRIGGGEDEFGRETAGRPVKAGLATSLGFGHVSGFVALVHPGAFEAAVAKADGEAALEAWRERANARLAAGQRHLEEGMMGRAALYEPIDNRRFREDHRGYDHHEVEKAMLLNPDARLGADGYYEA